SEQUENCE LISTING (172 sequences)

SEQ ID NO: 1 - protein, 8 aa, Homo sapiens
LVGDEVEL

SEQ ID NO: 2 - protein, 8 aa, Homo sapiens
ISPGKNAT

SEQ ID NO: 3 - protein, 8 aa, Homo sapiens
RNGKDQDG

SEQ ID NO: 4 - protein, 9 aa, Homo sapiens
QDGDQAPEY

SEQ ID NO: 5 - protein, 9 aa, Homo sapiens
PEYRGRTEL

SEQ ID NO: 6 - protein, 9 aa, Homo sapiens
TELLKDAIG

SEQ ID NO: 7 - protein, 9 aa, Homo sapiens
AIGEGKVTL

SEQ ID NO: 8 - protein, 10 aa, Homo sapiens
VTLRIRNVRF

SEQ ID NO: 9 - protein, 8 aa, Homo sapiens
VRFSDEGG

SEQ ID NO: 10 - protein, 7 aa, Homo sapiens
HSYQEEA

SEQ ID NO: 11 - protein, 7 aa, Homo sapiens
EEAAMEL

SEQ ID NO: 12 - protein, 7 aa, Homo sapiens
MELKVED

SEQ ID NO: 13 - protein, 7 aa, Homo sapiens
LEELRNP

SEQ ID NO: 14 - protein, 10 aa, Homo sapiens
TCRKLDELGS

SEQ ID NO: 15 - protein, 8 aa, Homo sapiens
MGQEPGVA

SEQ ID NO: 16 - protein, 9 aa, Homo sapiens
GVAKYRRAQ

SEQ ID NO: 17 - protein, 6 aa, Homo sapiens
AHRETT

SEQ ID NO: 18 - protein, 7 aa, Homo sapiens
TGTEKLI

SEQ ID NO: 19 - protein, 5 aa, Homo sapiens
SKTSA

SEQ ID NO: 20 - protein, 5 aa, Homo sapiens
ADARM

SEQ ID NO: 21 - protein, 8 aa, Homo sapiens
GGQKGRGS

SEQ ID NO: 22 - protein, 8 aa, Homo sapiens
GRGSRGQH

SEQ ID NO: 23 - protein, 7 aa, Homo sapiens
HSLERVC

SEQ ID NO: 24 - protein, 6 aa, Homo sapiens
GHPDKF

SEQ ID NO: 25 - protein, 9 aa, Homo sapiens
CTERHRHVD

SEQ ID NO: 26 - protein, 9 aa, Homo sapiens
RHVDCSGRN

SEQ ID NO: 27 - protein, 7 aa, Homo sapiens
SNNRLES

SEQ ID NO: 28 - protein, 8 aa, Homo sapiens
KLLDKSDT

SEQ ID NO: 29 - protein, 8 aa, Homo sapiens
DVSKNMLE

SEQ ID NO: 30 - protein, 7 aa, Homo sapiens
WSCDHKQ

SEQ ID NO: 31 - protein, 6 aa, Homo sapiens
SLKEHN

SEQ ID NO: 32 - protein, 10 aa, Homo sapiens
KVTKIPKQYR

SEQ ID NO: 33 - protein, 9 aa, Homo sapiens
KQYRTKETT

SEQ ID NO: 34 - protein, 6 aa, Homo sapiens
LSKDTT

SEQ ID NO: 35 - protein, 5 aa, Homo sapiens
STDKA

SEQ ID NO: 36 - protein, 10 aa, Homo sapiens
PEDTSTETIN

SEQ ID NO: 37 - protein, 10 aa, Homo sapiens
TETINSHEAA

SEQ ID NO: 38 - protein, 7 aa, Homo sapiens
SSTKSSP

SEQ ID NO: 39 - protein, 9 aa, Homo sapiens
WREETTTNV

SEQ ID NO: 40 - protein, 276 aa, Homo sapiens
GLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 41 - protein, 17 aa, Homo sapiens
SIGSLCADARMYGVLPW

SEQ ID NO: 42 - protein, 19 aa, Homo sapiens
AHLVQPSQADARMLMYQHQ

SEQ ID NO: 43 - protein, 19 aa, Homo sapiens
AHLVQPSQADARMLMYQHQ

SEQ ID NO: 44 - protein, 17 aa, Homo sapiens
QSIAFPSKTSASIGSLC

SEQ ID NO: 45 - protein, 16 aa, Homo sapiens
HNDGEESKTSAQVFEH

SEQ ID NO: 46 - protein, 17 aa, Homo sapiens
KESPPPSKTSAAAQLDE

SEQ ID NO: 47 - protein, 17 aa, Homo sapiens
TSTPQTSKTSAATSSTT

SEQ ID NO: 48 - protein, 17 aa, Homo sapiens
AYTYDRSKTSARIYPTY

SEQ ID NO: 49 - protein, 17 aa, Homo sapiens
SMVKIPSKTSAKYLEKK

SEQ ID NO: 50 - protein, 17 aa, Homo sapiens
TYKVATSKTSAKLKVEA

SEQ ID NO: 51 - protein, 16 aa, Homo sapiens
KDTTFTSTDKAFVPYP

SEQ ID NO: 52 - protein, 17 aa, Homo sapiens
HDCVQLSTDKAGVVAEY

SEQ ID NO: 53 - protein, 17 aa, Homo sapiens
TTHLPFSTDKAIAIVVA

SEQ ID NO: 54 - protein, 17 aa, Homo sapiens
NATFTVSTDKAQRHFGY

SEQ ID NO: 55 - protein, 17 aa, Homo sapiens
ASWTIISTDKAEYTFYE

SEQ ID NO: 56 - protein, 17 aa, Homo sapiens
TSITLISTDKAEQVNTE

SEQ ID NO: 57 - protein, 17 aa, Homo sapiens
SEEERGSTDKARHFLES

SEQ ID NO: 58 - protein, 17 aa, Homo sapiens
LGIQNPSTDKAPKGPTG

SEQ ID NO: 59 - protein, 11 aa, Homo sapiens
PGAVASSTDKA

SEQ ID NO: 60 - protein, 17 aa, Homo sapiens
PLCIGVSTDKASMEVRY

SEQ ID NO: 61 - protein, 17 aa, Homo sapiens
HDCVQLSTDKAGVVAEY

SEQ ID NO: 62 - protein, 247 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLVFLCLQYRLRGKLRAEIENLHRTFDPHFLRVPCWKITLFVIVPVLGPLVALIICYNWLHRRLAGQFLEELRNPF

SEQ ID NO: 63 - protein, 208 aa, Homo sapiens [sequence omitted]

SEQ ID NO: 64 - protein, 224 aa, Homo sapiens [sequence omitted]

SEQ ID NO: 65 - protein, 131 aa, Homo sapiens [sequence omitted]

SEQ ID NO: 66 - protein, 252 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLVFLCLQYRLRGKLRAEIENLHRTFDPHFLRVPCWKITLFVIVPVLGPLVALIICYNWLHRRLAGQFLEELLFHLEALSG

SEQ ID NO: 67 - protein, 213 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLVFLCLQYRLRGKLRAEIENLHRTFGQFLEELLFHLEALSG

SEQ ID NO: 68 - protein, 229 aa, Homo sapiens [sequence omitted]

SEQ ID NO: 69 - protein, 206 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLVFLCLQYRLRGKLRAEIENLHRTFLFHLEALSG

SEQ ID NO: 70 - protein, 203 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLVFLCLQYRLRGKLRAEIENLHRTFGKFRHV

SEQ ID NO: 71 - protein, 5 aa, Homo sapiens
AHRET

SEQ ID NO: 72 - protein, 5 aa, Homo sapiens
FDFPD

SEQ ID NO: 73 - protein, 5 aa, Homo sapiens
NCPEL

SEQ ID NO: 74 - protein, 5 aa, Homo sapiens
EANGH

SEQ ID NO: 75 - protein, 5 aa, Homo sapiens
GTVLR

SEQ ID NO: 76 - protein, 5 aa, Homo sapiens
AYGQD

SEQ ID NO: 77 - protein, 5 aa, Homo sapiens
YGQDN

SEQ ID NO: 78 - protein, 5 aa, Homo sapiens
NVTVN

SEQ ID NO: 79 - protein, 5 aa, Homo sapiens
VTVNE

SEQ ID NO: 80 - protein, 5 aa, Homo sapiens
EFVYS

SEQ ID NO: 81 - protein, 5 aa, Homo sapiens
FVYSE

SEQ ID NO: 82 - protein, 5 aa, Homo sapiens
VYSER

SEQ ID NO: 83 - protein, 5 aa, Homo sapiens
LLGLR

SEQ ID NO: 84 - protein, 5 aa, Homo sapiens
LGLRG

SEQ ID NO: 85 - protein, 5 aa, Homo sapiens
MDHAR

SEQ ID NO: 86 - protein, 5 aa, Homo sapiens
PRHRD

SEQ ID NO: 87 - protein, 5 aa, Homo sapiens
EGQRP

SEQ ID NO: 88 - protein, 5 aa, Homo sapiens
GRASD

SEQ ID NO: 89 - protein, 5 aa, Homo sapiens
GGRDS

SEQ ID NO: 90 - protein, 5 aa, Homo sapiens
DSRSG

SEQ ID NO: 91 - protein, 5 aa, Homo sapiens
PMARR

SEQ ID NO: 92 - protein, 5 aa, Homo sapiens
PMARR

SEQ ID NO: 93 - protein, 5 aa, Homo sapiens
YKDSH

SEQ ID NO: 94 - protein, 5 aa, Homo sapiens
KDSHH

SEQ ID NO: 95 - protein, 5 aa, Homo sapiens
DSHHP

SEQ ID NO: 96 - protein, 5 aa, Homo sapiens
SHHPA

SEQ ID NO: 97 - protein, 5 aa, Homo sapiens
QDENP

SEQ ID NO: 98 - protein, 5 aa, Homo sapiens
YKSAH

SEQ ID NO: 99 - protein, 5 aa, Homo sapiens
AHKGF

SEQ ID NO: 100 - protein, 5 aa, Homo sapiens
GFKGV

SEQ ID NO: 101 - protein, 5 aa, Homo sapiens
SGSPM

SEQ ID NO: 102 - protein, 5 aa, Homo sapiens
GSPMA

SEQ ID NO: 103 - protein, 5 aa, Homo sapiens
GGSPV

SEQ ID NO: 104 - protein, 5 aa, Homo sapiens
LSRPS

SEQ ID NO: 105 - protein, 5 aa, Homo sapiens
SRPSL

SEQ ID NO: 106 - protein, 5 aa, Homo sapiens
QAPEY

SEQ ID NO: 107 - protein, 5 aa, Homo sapiens
VTLRI

SEQ ID NO: 108 - protein, 5 aa, Homo sapiens
HSYQE

SEQ ID NO: 109 - protein, 5 aa, Homo sapiens
AAMEL

SEQ ID NO: 110 - protein, 5 aa, Homo sapiens
KTGQF

SEQ ID NO: 111 - protein, 5 aa, Homo sapiens
TGQFL

SEQ ID NO: 112 - protein, 5 aa, Homo sapiens
HRTFE

SEQ ID NO: 113 - protein, 5 aa, Homo sapiens
HRTFD

SEQ ID NO: 114 - protein, 5 aa, Homo sapiens
KTGQF

SEQ ID NO: 115 - protein, 5 aa, Homo sapiens
TGQFL

SEQ ID NO: 116 - protein, 5 aa, Homo sapiens
RHVDC

SEQ ID NO: 117 - protein, 5 aa, Homo sapiens
HVDCS

SEQ ID NO: 118 - protein, 5 aa, Homo sapiens
VSKNM

SEQ ID NO: 119 - protein, 5 aa, Homo sapiens
SKNML

SEQ ID NO: 120 - protein, 5 aa, Homo sapiens
WSCDH

SEQ ID NO: 121 - protein, 5 aa, Homo sapiens
CDHKQ

SEQ ID NO: 122 - protein, 5 aa, Homo sapiens
IPKQY

SEQ ID NO: 123 - protein, 5 aa, Homo sapiens
PKQYR

SEQ ID NO: 124 - protein, 5 aa, Homo sapiens
STETI

SEQ ID NO: 125 - protein, 5 aa, Homo sapiens
ETINS

SEQ ID NO: 126 - protein, 5 aa, Homo sapiens
TINSH

SEQ ID NO: 127 - protein, 9 aa, Homo sapiens
FTISRDNAE

SEQ ID NO: 128 - protein, 10 aa, Homo sapiens
PASPLPEDLQ

SEQ ID NO: 129 - protein, 8 aa, Homo sapiens
SYRQRAQK

SEQ ID NO: 130 - protein, 8 aa, Homo sapiens
DDTNHERE

SEQ ID NO: 131 - protein, 9 aa, Homo sapiens
KAFKKELRQ

SEQ ID NO: 132 - protein, 8 aa, Homo sapiens
DVLKKSYS

SEQ ID NO: 133 - protein, 9 aa, Homo sapiens
LRQEKGGSR

SEQ ID NO: 134 - protein, 9 aa, Homo sapiens
VGELSRGKL

SEQ ID NO: 135 - protein, 9 aa, Homo sapiens
FDFPDELRP

SEQ ID NO: 136 - protein, 7 aa, Homo sapiens
NCPELRP

SEQ ID NO: 137 - protein, 9 aa, Homo sapiens
GRLREDEGT

SEQ ID NO: 138 - protein, 8 aa, Homo sapiens
PTREANGH

SEQ ID NO: 139 - protein, 8 aa, Homo sapiens
AYGQDNRT

SEQ ID NO: 140 - protein, 8 aa, Homo sapiens
CAAARDTV

SEQ ID NO: 141 - protein, 5 aa, Homo sapiens
SNPEP

SEQ ID NO: 142 - protein, 8 aa, Homo sapiens
YESERRLG

SEQ ID NO: 143 - protein, 8 aa, Homo sapiens
RLGSERRL

SEQ ID NO: 144 - protein, 9 aa, Homo sapiens
RGEPPELDL

SEQ ID NO: 145 - protein, 8 aa, Homo sapiens
DLGKRPTK

SEQ ID NO: 146 - protein, 8 aa, Homo sapiens
PTKDSYTL

SEQ ID NO: 147 - protein, 8 aa, Homo sapiens
PRHRDTGI

SEQ ID NO: 148 - protein, 8 aa, Homo sapiens
FGGDRGAP

SEQ ID NO: 149 - protein, 8 aa, Homo sapiens
PQKSHGRT

SEQ ID NO: 150 - protein, 8 aa, Homo sapiens
GRTQDENP

SEQ ID NO: 151 - protein, 9 aa, Homo sapiens
RQPRPRPEV

SEQ ID NO: 152 - protein, 9 aa, Homo sapiens
LRGPGASRG

SEQ ID NO: 153 - protein, 7 aa, Homo sapiens
SPVKASR

SEQ ID NO: 154 - protein, 7 aa, Nicotiana tabacum
DDSNHER

SEQ ID NO: 155 - protein, 7 aa, Neurospora crassa
KAFKKEL

SEQ ID NO: 156 - protein, 7 aa, Oryza sativa
DVLKKAY

SEQ ID NO: 157 - protein, 6 aa, Fusobacterium nucleatum
LRQEKG

SEQ ID NO: 158 - protein, 8 aa, Pseudomonas aeruginosa
VGEFSRGK

SEQ ID NO: 159 - protein, 6 aa, Pseudomonas aeruginosa
HRDTGI

SEQ ID NO: 160 - protein, 6 aa, Oryza sativa
FGGDRG

SEQ ID NO: 161 - protein, 6 aa, Clostridium perfringens
QKSHGR

SEQ ID NO: 162 - protein, 7 aa, Bacillus subtilis
RSQDENP

SEQ ID NO: 163 - protein, 6 aa, Salmonella enteritidis
PRPRPE

SEQ ID NO: 164 - protein, 8 aa, Pseudomonas aeruginosa
LRGPGATR

SEQ ID NO: 165 - protein, 7 aa, Pinus taeda
SPVKATR

SEQ ID NO: 166 - protein, 5 aa, Leptospira interrogans
DEVEL

SEQ ID NO: 167 - protein, 6 aa, Pseudomonas aeruginosa
RGRTEL

SEQ ID NO: 168 - protein, 6 aa, Pseudomonas putida
TERHRH

SEQ ID NO: 169 - protein, 6 aa, Saccharomyces cerevisiae
SNNRLE

SEQ ID NO: 170 - protein, 6 aa, Mycoplasma pneumoniae
SLKEHN

SEQ ID NO: 171 - protein, 6 aa, Staphylococcus aureus
WREETT

SEQ ID NO: 172 - protein, 6 aa, Homo sapiens
LSRPSL

(19) United States
(12) Patent Application Publication
(10) Pub. No.: US 2005/0272097 A1
(43) Pub. Date: Dec. 8, 2005

(54) Methods and compositions for detecting and treating autoimmune diseases

(76) Inventor: Emanuel Calenoff, Alameda, CA (US)

Correspondence Address: BARNES & THORNBURG, P.O. BOX 2786, CHICAGO, IL, 60690-2786, US

(21) Appl. No.: 11/062,186

(22) Filed: Feb. 18, 2005

Related U.S. Application Data
(60) Provisional application No. 60/546,062, filed on Feb. 18, 2004.
(60) Provisional application No. 60/545,980, filed on Feb. 18, 2004.

(51) Int. Cl. G01N 33/53; G01N 33/567

(52) U.S. Cl. 435/7.2

(57) ABSTRACT

Diseases caused by or affected by specific antibodies and/or T lymphocytes that complex with self-molecules in a subject are detected by identifying antigen specific antibodies and/or effector T lymphocytes against the antigen, in a biological fluid of an affected subject. This identification opens up treatment possibilities, for example, by desensitization.

### BACKGROUND

This application claims priority from U.S. Provisional Application Ser. Nos. 60/546,062 and 60/545,980, both filed Feb. 18, 2004.

Diseases caused by or affected by specific antibodies and/or T lymphocytes that complex with self-molecules in a subject are detected by identifying antigen specific antibodies and/or effector T lymphocytes against the antigen, in a biological fluid of an affected subject. This identification opens up treatment possibilities, for example, by desensitization.

Multiple sclerosis (MS) is a debilitating and sometimes fatal disease of the central nervous system (CNS) whose pathological pathway remains unresolved. The principal characteristics of MS are local inflammation of individually varying regions of the brain and/or spinal cord followed by variable destruction of myelin and denuded axons. The inflammation is reported to be caused by an autoimmune process. Whether that process is cellular, humoral, or a combination of both remains unresolved.

The immune system is a complicated network of cells and cell components that normally work to defend the body and eliminate infections caused by bacteria, viruses, and other invading microorganisms. If a person has an autoimmune disease, the immune system mistakenly attacks self, targeting the cells, tissues and organs of a person's own body. A collection of immune system cells and molecules at a target site is broadly referred to as inflammation.

Multiple Sclerosis

Multiple Sclerosis (MS) is characterized by the proliferation of autoreactive immune cells, their primary origin within or entry into the central nervous system (CNS) with subsequent perivascular inflammation and release of inflammatory cytokines, patchy demyelination and axonal injury/loss. Though cell-mediated aspects of immune injury have been emphasized in the pathogenesis of MS, the potential role of antibody-mediated injury is also recognized.

The potential role of serum anti-myelin IgG, and to some extent IgM, has been investigated but results are conflicting, which seems attributable at least in part to different assay systems. Serum anti-myelin IgE has not yet been studied. The potential roles of autoreactive IgE and the allergic system in CNS demyelinating disease have not been extensively explored to date.

The need for a diagnostic test(s) in MS is self-evident. Despite advances in understanding of and therapeutic options for multiple sclerosis, this disease remains a syndrome of unknown etiology(ies) without a definitive, front-end diagnostic tests. Though neuroimaging plays an important role, diagnosis is made largely on clinical grounds and requires that conditions that can mimic multiple sclerosis be excluded. It is generally believed that a viral trigger may be causal, with targeted damage being mediated by lymphocytes through the process of molecular mimicry, but this has not been proved, and it has been suggested that multiple sclerosis may in fact have multiple causes that reflect the heterogeneity of the disease clinically, radiographically, and pathologically. Much of our understanding of the immunopathogenesis of MS is based on animal models of CNS demyelinating disease.

Immunopathogenesis of MS:

Genetic factors of MS are most likely polygenic, in that genetic regions thus far implicated include a number of immunological genes such as the major histocompatibility regions. MS may be influenced by environment, though the actual trigger(s) has remained elusive. It is generally assumed that the trigger might be an infectious agent that instigates an appropriate immune attack, but that through the process of molecular mimicry, an autoimmune attack against central nervous system target proteins ensues. By and large, evidence favors an attack against myelin proteins, and a number of candidate myelin and neural proteins have been shown to trigger inflammatory demyelination in animal models. While many infectious triggers have been proposed in MS, none has yet proved to be causal in MS.

BBB disruption is a consistent early finding in MS and EAE lesion evolution. Most disruptions are found adjacent to active MS lesions and reflect the inflammatory component of this disease. Factors that can interrupt integrity of the BBB are many and include chemokines, cytokines and blood-borne histamine, though vascular and immune cell adhesion molecule expression are also important. It is conceivable that histamine release by peripheral mast cells might play an important role. Mast cells originate in the bone marrow from the same pluripotential stem cell (CD34+) as basophils. Mast cells subsequently are distributed to tissues via blood as non-granualated mononuclear cells. Within tissues, mast cells continue to differentiate, develop granules, and have a lifespan ranging from weeks to months.

Four pathological subtypes associated with lesions from biopsied or autopsied MS brains was reported suggesting that patterns I and II are defined by typical perivenular demyelinating lesions, whereas types III and IV, which appear to be less common, suggest oligodendrocyte dystrophy. Pattern II is distinguished from pattern I in that focal deposits of Ig (IgG) plus complement are seen in pattern II only. It is worth noting, however, that other Ig subtypes (including IgE) were not examined. It remains to be demonstrated whether such pathological heterogeneity is specific for distinct disease patterns that occur in different subpopulations of MS patients, and whether a given pattern remains consistent within an individual patient over time. Pathological reevaluation of brain biopsies from MS patients prior to treatment with plasmapheresis shows that patients responsive to treatment have a type II pathology.

Mast cells, the toxic reservoirs of IgE-mediated disease, have not been universally described on biopsies or autopsies of MS CNS tissue. However, it is conceivable that they are of low number, are a transient effector in an immune response dominated by other cell-mediated and humoral mechanisms, or might not be recognized without specific stains. When actively sought, mast cells have been described in the brains of multiple sclerosis patients, and it is plausible that they might play a destructive role in the lesions of at least some MS patients, either as an early trigger or an ongoing mediator through the release of histamine (systemic or local), proteases, and immune-modulating cytokines, chemokines, leukotrienes and/or prostaglandins.

Cell-Mediated vs. Humoral Mechanisms in MS

Controversy exists as to whether the primary immunopathogenic mechanism in MS is T or B-cell mediated, and some studies of cells and CSF antibodies indicate overlapping B- and T cell immunodominant MBP epitopes; in any case, it is clear that multiple immune cell types are responsible. Over the past couple of decades, research has focused on the role of the T cell (TH1) in this disease, which has been fueled by the demonstration of the T-cell's importance in experimental autoimmune encephalomyelitis (EAE). Pathologically, the presence of plasma cells and Ig in active and chronic MS lesions is known. Ig-associated receptor mediated phagocytosis of myelin remnants by macrophages and complement activation within the CNS, the intrathecal synthesis of Ig, and the detection of clonally expanded B-cells indirectly support a pathogenic role of antibody and MS lesion evolution. Using gold-conjugated myelin peptides, it has been demonstrated that antigenic specific autoantibodies are present in MS lesions as well as lesions in EAE, strongly suggesting that antibody-mediated myelin destruction occurs in MS. While EAE can be induced by passage of autoreactive T-cells, autoantibodies can nonetheless combine to cause greater injury.

A central unanswered question is whether autoreactive Igs can cause CNS injury or simply represents an epiphenomenon occurring subsequent to CNS injury, or both. Potential myelin target antigens are many, including: myelin-oligodendrocyte glycoprotein (MOG), myelin basic protein (MBP), proteolipid protein (PLP), claudin-11 (oligodendrocytes-specific protein, OSP), 2′,3′-cyclic nucleotide 3′-phosphodiesterase (CNP) and oligodendrocyte-myelin glycoprotein (OMgp).

IgE is much less prevalent than other Ig subtypes, and as a result has not been tested in MS, whereas more abundant subtypes have. Reasons for this are likely twofold: 1) the pathogenic potential of anti-myelin IgE is not appreciated; 2) measuring an IgE response is technically difficult, given that IgE makes up a small proportion of all Igs. If anti-myelin protein IgE plays a pathogenic role in MS, it is possible that mast cells may also be involved in disease mechanisms. Particularly, if an epitope is closely repeated (between 40 to 100 angstroms), the potential for bivalent IgE binding and mast cell degranulation exists.

Experimental CNS Myelin Injury and Humoral Mechanisms:

The best accepted animal model of MS is experimental autoimmune (formerly, allergic) encephalomyelitis (EAE). As already noted, the TH 1 cell has been a focus of this model, although B-cells, antibodies and other immune cells are likely to play important roles in immune-mediated demyelination. For example, anti-MOG antibodies induce extensive demyelination in T-cell mediated EAE.

Connection Between Allergies and MS:

To date, there is little published for or against an increased prevalence of allergies in MS patients, and what there is, comes to conflicting conclusion. No study has examined serological evidence for allergy in MS in a blinded fashion. While an environmental trigger has been suspected based on epidemiological data, no known exogenous agent has been unambiguously identified to date.

While allergies are by themselves not sufficient to trigger MS, they might play a pathogenic role in MS. Allergy could serve as an environmental trigger to derange the immune system, and through the process of molecular mimicry, a heightened B-cell response with IgE autoantibodies might result; this response and secondary responses of B- and T-cells might lead to autoimmune attack of target CNS antigens. It is also conceivable that allergy might play a secondary role. That is, in individuals with MS, a coexistent allergy tendency could result in heightened generation of autoreactive IgE. Finally, allergies may exert an effect in breaking down the BBB (through mast cell degranulation), allowing pathogenic T- and B-cells to enter the CNS.

### SUMMARY

A method for detecting an autoimmune disease such as multiple sclerosis and rheumatoid arthritis, detects antibodies that form an immunocomplex with self-molecules in a subject as well as detecting disease-specific T lymphocytes. The method includes the steps of first identifying an antigen molecule comprising one or more epitopes on a self-molecule of a subject, wherein the antigen molecule is specific to the disease. The self-molecule is generally a protein or a glycoprotein. The antigen molecule does not contain epitopes that do not participate in the autoimmune disease process to avoid the formation of a non-specific immunocomplex. The antigen molecule is generally a peptide, a protein, or three-dimensional protein fragment. For example, the self-molecule of multiple sclerosis is a constituent of an oligodendrocyte or central nervous system myelin. After the antigen is identified, a biological fluid of the subject is measured for disease-specific antibodies or T lymphocytes, which form an immunocomplex with the epitopes on the antigen molecule. The presence of the specific antibodies or lymphocytes is an indication of the disease. After one or more disease-specific epitopes on a self molecule of an autoimmune patient are identified, the level of the specific immunoglobulin against such epitopes in a biological fluid of a patient is quantitated by an immunoassay. Based on the specific immunoglobulin level, a desensitization treatment is established by administering to the patient a therapeutic antigen comprising specific epitopes. The desensitization treatment is designed to down-regulate the immune process responsible for producing the immunoglobulin or helper T lymphocytes, thus ameliorating the symptom or physical finding of the autoimmune disease.

In particular, a method for detecting a disease caused or affected by antibodies that complex with self-molecules in a subject includes the steps of:
(a) identifying an antigen molecule that includes one or more epitopes on a self molecule of a subject, wherein the antigen molecule is specific to the disease and each epitope has five to six contiguous amino acids, the sequence of which confers three dimensional target specificity for an individual antibody; and
(b) detecting a specific IgA, IgD, IgE, IgG, and/or IgM antibody in a biological fluid of the subject, wherein said antibody forms an immunocomplex with said one or more epitopes on the antigen molecule, whereby said autoimmune disease is detected; the biological fluid may be plasma, serum, or whole blood.

A specific IgA antibody is IgA1 or IgA2. The specifically bound IgA2 antibody is pathologic because it is able to fix complement. The specifically bound IgA1 antibody is protective because it is able compete with co-existing, harmful antibodies or T lymphocytes for specific epitope binding or by supplanting specific epitope binding by harmful antibodies or lymphocytes.

A specific IgG antibody may also be IgG1, IgG2, IgG3 and/or IgG4. When the specifically bound IgG antibody is IgG₁ and/or IgG₃, it is pathologic because it is able to fix complement. The specifically bound IgG2 and/or IgG4 antibody is protective because it is able to compete with co-existing, harmful antibodies or T lymphocytes for specific epitope binding or by supplanting specific epitope binding by harmful antibodies or lymphocytes.

A specifically bound IgM antibody is pathologic because it is able to fix complement. The specifically bound antibody is able to serve as an opsonizing agent for cellular autoimmunity.

Suitable diseases include autoimmune diseases, neurological diseases, or psychiatric diseases. An autoimmune disease is multiple sclerosis wherein the self-molecule is a constituent of an oligodendrocyte or central nervous system myelin, citrullinated myelin basic protein, Claudin-11, a myelin basic protein (MBP), a myelin oligodendrocyte basic protein (MOBP), a myelin oligodendrocyte glycoprotein (MOG), or an oligodendrocyte myelin glycoprotein (OMgp).

For example, MS patient and control sera were examined for measurable IgE against peptides derived from four myelin proteins: OMgp, MOG, PLP, and OSP (FIG. 1). Selected peptides represent linear regions on the protein surface that are likely to be exposed, based on hydrophilicity analysis and known structures. IgE against self-antigen was found in biological fluids of autoimmune disease patients. IgE-driven, mast cell degranulation is a component of an autoimmune disease. Diseases caused by or affected by IgE antibodies that complex with self-molecules in a subject can be detected by first identifying a disease-specific antigen molecule comprising one or more epitopes on a self-molecule of a subject. The specific IgE antibody level in the biological fluid of a subject that forms an immunocomplex with the antigen can then be measured by an immunoassay. The presence of the specific IgE is an indication of the disease. Myelin-specific IgE is present in the systemic circulation, but is absent in the cerebrospinal fluid, which indicates that most IgE production is external to the blood-brain barrier (BBB).

IgE-driven, mast cell degranulation is a component of an autoimmune disease. In the method disclosed, IgE forms an immunocomplex with one or more epitopes in the form of an IgE dimer, IgE trimer, or IgE oligomer; the immunocomplex is able to induce mast cell degranulation; and the mast cell degranulation initiates, sustains, or augments a specific disease process.

The distance between each IgE molecule of the IgE dimer, IgE trimer, or IgE oligomer complex is between 40 to 100 angstroms. Each IgE molecule of the IgE dimer, IgE trimer, or IgE oligomer complex is spatially arranged so as to adequately form an immunocomplex with Fc RI receptors on a mast cell such that mast cell degranulation is induced.

The Fc portion of the IgE molecule has undergone a structural modification to facilitate its immunocomplex formation with Fc RI receptors on a mast cell.

A specific IgE antibody is detected by an immunoassay that includes the steps of:

(a) reacting the biological fluid with a solid phase immobilized with the antigen molecule to form an immunocomplex;

(b) removing unbound materials from the solid phase;

(c) reacting the solid phase of (b) with an anti-IgE antibody labeled with a reporter molecule;

(d) removing unbound materials from the solid phase of (c); and

(e) detecting a signal generated from the reporter molecule on the solid phase of (d).

The reporter molecule may be a radioisotope, an enzyme, a fluorescent molecule, or a chemiluminescent molecule.

A method for detecting a disease caused or affected by T lymphocytes that complex with self-molecules in a subject include the steps of:

(a) identifying an antigen molecule including one or more epitopes on a self molecule of a subject, wherein the antigen molecule is specific to the disease and each epitope comprises five to six contiguous amino acids the sequence of which confers linear target specificity for an individual lymphocyte;

(b) the specific T lymphocyte forms an immunocomplex with an epitope on the antigen molecule; and

(c) detecting specific T lymphocytes in a biological fluid of the subject, wherein said lymphocytes are stimulated to proliferate in the presence of their corresponding specific epitope, whereby the autoimmune disease is detected.

The T lymphocyte is a helper T lymphocyte or a suppressor T lymphocyte.

A plurality of immunogenic peptides of a self molecule, said peptides which produce a disease or condition specific immune response in a host, wherein the target protein is causative of, or associated with, a targeted disease or condition, and said peptides comprise the following structure:
(a) from 5 to 10 amino acids in length;
(b) an amino acid sequence which is identical to a contiguous amino acid peptide region of the sequence of a protein designated the target protein;
(c) a net hydrophilic structure as determined by the amino acid sequence of the peptide, said structure located on the surface of the target protein;
(d) an amino acid net sequence homology of 50 percent or less as compared with contiguous amino acid sequences of a comparative protein;
(e) an amino acid sequence wherein no more than three contiguous amino acids are identical to contiguous amino acids of the comparative protein matched for overall homology; and
(f) an antigenic profile which elicits an immune response specific for the target protein as determined by results of immune cell proliferation assays or immunoassays of targeted disease or condition positive biological fluids compared to disease or condition negative biological fluids.

A pharmaceutical composition comprising the peptide ADARM or other peptides shown in FIG. 4.

A pharmaceutical composition comprising a plurality of immunogenic peptides as produced by disclosed methods.

An immunogenic composition capable of inducing a mammal to produce antibodies specific for an epitope on a target protein, wherein the immunogenic composition comprises a plurality of peptides of claim 20.

A method for treating an autoimmune disease in a subject includes the steps of:
(a) identifying one ore more epitopes on a self molecule of a subject that are specific to an autoimmune disease;
(b) quantifying specific immunoglobulin complexes with one or more epitopes, an epitope comprising five to six contiguous amino acids the sequence of which confers three dimensional target specificity for an individual antibody;
(c) establishing a starting dose of a therapeutic antigen for desensitizing treatment, wherein the therapeutic antigen includes one or more epitopes; and the dose is inversely proportional to the quantitated immunoglobulin level of (b);
(d) administering to the subject the starting dose (c) of the therapeutic antigen; and
(e) administering to the subject an incremental dose of the therapeutic antigen over a period of time, to a level sufficient to down-regulate an immune process responsible for producing the immunoglobulin; whereby the symptom or physical finding of autoimmune disease is ameliorated.

Again, immunoglobulin includes IgA, IgD, IgE, IgG, or IgM. Biological fluid include plasma, serum, cerebrospinal fluid, or whole blood. The self-molecule includes a protein or a glycoprotein. The therapeutic antigen binds to the specific immunoglobulin the complexing of which induces desensitization that is commonly practiced for other conditions such as respiratory allergy. The basic immunoregulatory process behind desensitization is also commonly known as tolerization. The therapeutic antigen does not contain epitopes that do not participate in the autoimmune disease process. One or more epitopes are located on a peptide, a protein, or a three-dimensional protein fragment. The therapeutic antigen is administered intradermally, subcutaneously, orally, rectally, or topically. The topical administration is via inhalation or skin administration, the intradermal or subcutaneous administration is by injection.

A method for treating an autoimmune disease in a subject includes the steps of:
(a) identifying one or more epitopes on a self molecule of a subject that are specific to an autoimmune disease, wherein an epitope comprises five to six contiguous amino acids the sequence of which confers linear target specificity for an individual T helper lymphocyte;
(b) identifying the presence of epitope-specific T helper lymphocytes in the subject's blood or other bodily reservoir by epitope-specific proliferation studies or by other studies;
(c) testing the expanded, epitope-specific T lymphocytes in cytoxicity studies directed against oligodendrocytes or other neural cells in order to confirm specific pathology;
(d) quantifying a specific immunoglobulin level in a biological fluid of the subject, wherein said immunoglobulin complexes with the epitope(s) specifically recognized by the T lymphocyte;
(e) establishing a starting dose of a therapeutic antigen for desensitizing treatment, wherein said therapeutic antigen comprises said one or more epitopes; and said dose is inversely proportional to the quantitated immunoglobulin level of (d);
(f) administering to the subject the starting dose (c) of the therapeutic antigen; and administering to the subject an incremental dose of the therapeutic antigen over a period of time, to a level sufficient to down-regulate an immune process responsible for producing the epitope-specific T lymphocyte; whereby the symptom or physical finding of autoimmune disease is ameliorated.

### BRIEF DESCRIPTION OF THE DRAWINGS

FIG. 1A shows IgE Serum positivity against myelin protein peptides for 32 RRMS patients (including 3 CIS patients, far left). A shaded box indicates IgE reactivity against that peptide, with titer of specific IgE indicated by the number within the box. Reactivity requires signal at least 2.0×SD above control peptide. In cases where peptide reactivity is unique to the tested MS population, boxes are stippled (suggesting disease-specific peptide sequence); in cases where peptide reactivity is also found in controls but at much lower levels (>2.0×SD), boxes are highlighted without stippling (suggesting disease-associated sequence). Serum from RRMS patients more often tested IgE positive against one or more allergens, as compared to PPMS patients or controls (bottom).

FIG. 1B shows serum IgE positivity against myelin protein peptides for 14 SPMS (left) and 7 PPMS patients. A shaded box indicates IgE reactivity against that peptide, with titer of specific IgE indicated by the number within the box. Reactivity requires signal at least 2.0×SD above control peptide. In cases where peptide reactivity is unique to the tested MS population, boxes are stippled (suggesting disease-specific peptide sequence); in cases where peptide reactivity is also found in controls but at much lower levels (>2.0×SD), boxes are highlighted without stippling (suggesting disease-associated sequence).

FIG. 1C shows serum IgE positivity against myelin protein peptides for 30 controls. A shaded box indicates IgE reactivity against that peptide, with titer of specific IgE indicated by the number within the box. Reactivity requires signal at least 2.0×SD above control peptide.

FIG. 2A shows the presence of pentameric peptide ADARM

FIG. 2B Presence of pentameric peptides (ADARM and SKTSA from PLP; STDKA from OMgp) in other human proteins. As shown, the sequence ADARM from PLP (IgE reactive in some patients) has a complete pentameric protein surface exposure not found on other human proteins with similar amino acid sequence representation whereas the more promiscuous IgE nonreactive surface peptides SKTSA (PLP) and STDKA (OMgp) are found in complete pentameric form on the surface of a variety of proteins. Disease-specific antigenic peptides are underrepresented among the pool of human proteins. Hydrophilic areas are shown boxed.

FIG. 3 Nine different MOG isoform sequences are depicted. The net hydrophilic amino acid sequences likely to represent protein surface areas are boxed (encircled). Interrupted box frames represent cytoplasmic localization whereas uninterrupted box frames represent externalized (outer myelin surface) protein portions. Sequences in stippled boxes represent functional epitopic sequences that found on few or no other human proteins. The early sequence LSRPSL likely represents a leader sequence component of each MOG protein.

FIG. 4 shows myelin protein pentamers representing specific humoral epitopes used for assay purposes.

### DETAILED DESCRIPTION

Autoimmune Diseases

The present methods and compositions are suitable for detecting a disease caused by, or affected by, IgE antibodies that complex with self-molecules in a subject, including an autoimmune disease, a neurological disease, and a psychiatric disease; particularly an autoimmune disease.

Autoimmune diseases suitable for detection by the present invention include multiple sclerosis, myasthenia gravis, autoimmune neuropathies (Guillain-Barré) and autoimmune uveitis, of which nervous system is the target organ; autoimmune hemolytic anemia, pernicious anemia, and autoimmune thrombocytopenia, of which blood is the target; temporal arteritis, anti-phospholipid syndrome, and vasculitides (such as Wegener's granulomatosis Behcet's disease), of which blood vessels are the target; Crohn's Disease, ulcerative colitis, primary bililary cirrhosis, and autoimmune hepatitis, of which gastrointestinal system is the target; Type 1 or immune-mediated diabetes mellitus, Grave's Disease, Hashimoto's thyroiditis, autoimmune oophoritis and orchitis, and autoimmune disease of the adrenal gland, of which endocrine glands are the target; rheumatoid arthritis, of which connective tissues (muscle, skeleton, tendons, fascia) are the target; psoriasis, dermatitis herpetiformis, pemphigus vulgaris, vitililgo, systemic lupus erythematosus, scleroderma, polymyositis, dermatomyositis, spondyloarthropathies (such as ankylosing spondylitis), and Sjogren's syndrome, of which skin is the target.

Use of short peptides, approximately 5-amino acids in length, has not been reported in specific assay construction. Instead, the main focus has been upon simulating three-dimensional molecular structure in order that the synthetic peptides mimic their native parent molecules, whereas the opposite condition is warranted in most cases: (a) when used as a T-cell immunogen, a linear peptide is appropriate as a two-dimensional structural representation; and (b) in representing specific epitopes, peptides that are 5-6 amino acids in length work well because they are able to flex adequately and in-fold precisely into the epitope binding domain of the specific measured antibody (with high affinity-avidity), whereas those of similar size that deviate in their amino acid sequence cannot. Peptides that are longer than 5-6 amino acids take fairly rigid, unpredictable 3D forms that often do not mimic their parent protein's three-dimensional structure. In our experience, these are less able to in-fold properly and become less so as their length increases.

Epitopes that follow the linear contour of their respective proteins are predictable when using molecular mapping technology. For epitopes that are purely three-dimensional, crossing over from one peptide strand to another, 5-6 amino acid length peptides can also suffice but must be selected for their functional mimicry.

Based on Ig (in particular IgE) mediated reactivity against neural components as an important element in MS pathology, antigen-specific desensitization, similar to conventional allergy desensitization, is a logical therapeutic approach. Interestingly, the milk protein butyrophilin possesses homology to MOG, and in serum obtained from MS patients, crossreactive antibodies have been identified, suggesting a role for molecular mimicry. If common inhalant or food allergens possess epitopes similar in structure to IgE-reactive sites on CNS proteins, treatment would comprise desensitization with the appropriate, available inhalant allergen solutions together with dietary elimination of IgE-reactive foods. A more specific therapeutic approach could entail construction of novel desensitizing agents comprising small Igreactive peptides or small protein fragments, alone or coupled to neutral carriers, akin to the experimental altered peptide ligand approach, but specific on an individual to individual basis, depending upon results from a peptide screening assay such as that described here.

Particular autoimmune diseases suitable for detection by the present methods and compositions are rheumatoid arthritis and multiple sclerosis because of the indication of mast cell presence within active MS lesions or in RA synovium.

Mast Cell Degranulation

The complex formation of self-antigen and IgE, which mediates mast cell degranulation, is an important component of autoimmune diseases.

A functional, mast cell-reactive antigen possesses multivalent and/or polyvalent IgE epitopes. Multivalent epitopes (different epitopes) require two or more individually reactive IgE antibodies for a proper complex formation. Patients possessing too few epitope-specific IgE types do not form a proper complex, thus do not undergo mast cell degranulation. In contrast, antigens with polyvalent epitopes (same repeated epitopes) can complex with a single, available antibody type. This is one basis of differentiation between subjects who develop autoimmune disease and those who do not. Another differentiating factor is the availability of a functional antigen lattice comprising two correctly spaced antigen molecules, each with one IgE-reactive epitope [i.e. myelin oligodendrocyte glycoprotein (MOG) in multiple sclerosis].

A further disease-differentiating factor is the site-specific localization of individual target proteins. Some proteins or their isomers are known to be variably expressed among different anatomical sites or are expressed in varying quantities. This would determine whether mast cell degranulation occurs in a particular location, and if so, how much degranulation takes place.

Multiple Sclerosis

Multiple sclerosis is a disease in which the immune system targets nerve tissues of the central nervous system (CNS). Multiple sclerosis is an autoimmune disease that is suitable for detection by the disclosed methods. Myelin-specific IgE is present in the systemic circulation, but is absent in the cerebrospinal fluid, which indicates that most IgE production is external to the blood-brain barrier (BBB). To affect CNS inflammation, systemically produced IgE must be able to pass through the BBB.

MS lesion (plaque) formation and subsequent neural recovery is a fluctuating process. Differences among MS patients exist as to plaque site distributions, degree and rate of damages to the affected neurons, frequency of inflammatory episodes, and degree and rate of neuronal recovery. Four disease models involving IgE-mediated mast cell degranulation to explain why MS occurs and its various pathological manifestations.

In one IgE-mediated disease model, an acute, localized opening of the BBB would allow systemic anti-neural IgE to make contact with CNS regions in proximity to the site of disruption. Degranulation of intrathecal mast cells by antigen-complexed IgE initiates and sustains local inflammation of oligodendrocyte cell bodies, projecting myelin, or denuded axons. Granule-contained enzymes such as tryptase directly disrupt target cells or tissues. Released chemotactic factors recruit or stimulate various immune cell types to supercede or augment the initial destructive process. Granule-released histamine continues to disrupt the BBB in proximity to the site of inflammation. A persistently open BBB allows entry of additional blood-borne IgE. The localized inflammatory process continues as long as specific target antigens and functioning mast cells remain available. Alternatively, an intrathercal autoimmune process can be induced by the initial mast cell inflammatory process and eventually supercede it.

In a second disease model, mast cell degranulation resulting from an acute BBB breakdown does not provide significant tissue damage but allows for a locally protracted BBB disruption and an influx of tissue-specific, non-IgE antibodies and/or antigen-sensitized immune cells. The non-IgE antibodies cause their principal damages by fixing complement. Extraneous antibodies also facilitate a localized, cellular immune process.

A third disease model incorporates elements of model 1 and model 2.

A fourth disease model incorporates elements of any of the first three models but function within the peripheral nervous system (PNS) by breeching the blood nerve barrier (BNB), alone or together with a compromised BBB. This model can explain why some MS patients experience severe peripheral nerve damage (have PNS involvement) and why others lack such pathology. It can also explain causation of some singular peripheral neuropathies.

Mast-cell degranulation based upon BBB penetration of CNS-specific, blood-borne IgE is an important component in MS. Degranulation may also prove important in other, poorly defined, neurological disorders, where systemic IgE is found to react with specific CNS or PNS locations other than those containing oligodendrocytes, CNS myelin, or enveloped axons.

Method for Preparing Self-Antigenic Peptides

Specific antigenic peptides that are epitopes of self-antigens of autoimmune diseases can be prepared and selected by any methods known to a person of skill in the art.

The antigenic peptides are preferably small, e.g. from 5 to about 100 amino acids in length. More preferred lengths of the peptides are from 5-6 amino acids or 5-10 amino acids, although peptides up to about 25 or to 100 amino acids in length are also within the scope of the invention. The peptides have a net hydrophilic structure located on the surface of a parent molecule (protein) from which they are derived or are synthesized from knowledge of the parent molecule. The peptides or fragments thereof include any variation in the amino acid sequence, whether by conservative amino acid substitution, deletion, or other processes. In an embodiment of the invention, more than one antigenic peptide (a plurality of antigenic peptides) can be used to enhance the discriminatory power of the immunoassay that quantitates the specific antibody level, thus improving the therapeutic procedures.

The steps outlined in Table 1 lead to the production of antigenic peptides suitable for the practice of the present invention.

TABLE 1
Steps in Obtaining Immuno-Specific Peptides Containing a Single
Epitope or a Small Number of Epitopes

Step 1: identify an autoimmune disease to be targeted for therapy.
Step 2: target a protein or glycoprotein (parent protein molecule)
causative of, or associated with, the autoimmune disease of step 1.
Step 3: obtain the amino acid sequence of the parent protein
molecule of step 2.
Step 4: map the hydrophilic peptide regions of the parent molecule by
analyzing the amino acid sequence of step 3 employing the rolling sum
analysis of 7 consecutive residues.
Step 5: select at least one peptide from 5-100 amino acids in length.
Step 6: compare the amino acid sequence of all known non-specific host
proteins (comparative proteins) for possible amino acid sequence
homology with the peptides of Step 5.
Step 7: select as candidate, functionally specific peptide antigens those
peptide sequences of Step 5 that comprise at least 5 contiguous amino
acids, the epitope, wherein no more than 4 contiguous amino acids within
each epitope are homologous to other protein sequences and wherein
several, overlapping epitopes may constitute unique peptide sequences
that are longer than 5 amino acids.
Step 8: reject those peptide sequences of Step 7 that have sequences of
4 or more contiguous amino acids which are identical to contiguous
sequences of the comparative proteins.
Step 9: identify the non-rejected peptide regions of step 8 and synthesize
structurally identical peptides to them for use a source antigen in step 10.
Step 10: determine immunospecificity of the synthetic peptides of step 9
by comparing immunoassay results on disease-positive biological fluids
with biological fluids from disease-negative individuals, for the presence
or absence of antibodies which specifically complex with the synthetic
peptides. Alternatively, use the synthetic peptides of Step 10 to compare
biological fluids from disease-positive organisms or individuals and
biological fluids from disease-negative organisms or individuals for the
presence or absence of immune cells which specifically complex with
the synthetic peptides and/or are stimulated by them.
Step 11: discard from consideration peptide sequences of Step 9 which
neither complex in a highly specific way with antibodies in disease-
positive biological fluids nor complex in a highly specific way with
and/or stimulate in a highly specific way immune cells from within a
disease positive biological fluid.
Step 12: peptides not discarded in Step 13 are suitable for practice of
the present invention for developing diagnostic and/or therapeutic products
or technologies.

To prepare antigenic peptides where an autoimmune disease is targeted, a tissue, or a constituent molecule of a tissue, which is known to be associated with the targeted disease, is first identified. Proteins from the tissue are selected from databases, e.g. the NIH gene bank, which is available on the Internet. These proteins are called “parent” proteins. Functionally specific peptide antigen candidates are identified from within the amino acid structure of each parent protein on the basis of being hydrophilic and therefore likely to be on the outer surface of the protein. The amino acid structures of the candidate antigens are then compared with the amino acid structures found in individual foreign (non-specific) proteins by using computer matching programs such as BLAST. Functionally specific antigenic peptides are selected on the basis of comprising at least 5 contiguous amino acids, the epitope, wherein no more than 4 contiguous amino acids within each epitope are homologous to other protein sequences and wherein several, overlapping epitopes may constitute unique peptide sequences that are longer than 5 amino acids.

Peptides that function as specific self-antigenic peptides for an autoimmune disease. Suitable peptides are five to one hundred amino acids in length, have a net hydrophilic structure. These peptides present an antigenic profile that elicits a highly specific, antibody-reactive immune response and/or highly specific, cellular immune response.

Self-Antigens of Multiple Sclerosis: Phase 1 Analysis

164 of 5-10 amino acid length peptides corresponding to nine myelin/oligodendrocyte proteins were analyzed as a potential source antigen for multiple sclerosis. Applicant has identified antigenic peptides corresponding to eight myelin/oligodendrocyte proteins: ceramide UDP-galactosyltransferase (CGT); 2′,3′-cyclic nucleotide 3′-phosphodiesterase (CNP); the CNS isomer of myelin-associated glycoprotein (L-MAG), myelin-associated oligodendrocyte basic protein (MOBP); myelin basic protein (MBP); myelin oligodendrocyte glycoprotein isomers (MOG); oligodendrocyte transmembrane protein (Claudin-11 or OSP); and oligodendrocyte myelin glycoprotein (OMG).

The IgE-reactive peptides of mylin/oligodendrocytes were analyzed using the BLAST search tool of the National Library of Medicine. The results show that most of the reactive peptides were significantly similar in structure to peptide regions of bacterial, food, fungal, or viral proteins, which are foreign proteins to human. These results may explain how specific IgE's against mylin/oligodendrocytes peptides are developed in patients even though mylin/oligodendrocytes proteins are behind the blood-brain barrier.

Patients possessing antibodies adversely reacting with a three-layer combination comprising myelin/oligodendrocyte surface antigens plus intracellular antigens plus axonal surface antigens are likely to suffer greater neural damage than patients with lesser reactive combinations. Patients possessing functional antibodies against outer membrane surface antigens are expected to suffer outer membrane disruption. If the disruption occurrs on the oligodendrocyte cell body, the oligodendrocyte and its projecting myelin will perish. The exposed regions of axon are susceptible to diminished neural conduction or to disruption by anti-axonal antibodies or specific cellular immune reactivity. If a subject possesses antibodies only against myelin membrane antigens, the oligodendrocyte body may not suffer damage if its projected myelin is only partially disrupted and has time to recover. Partial disruption is expected to occur only if the outer-most myelin layer is damaged in the absence of reaction against intracellular myelin proteins (i.e. against CNP, MBP, or MOBP) or against the cytoplasmic domains of transmembrane proteins (i.e. MAG, MOG).

Variations in neural pathology are expected from quantitative differences in available anti-neural IgE or other antibody isotypes. Patients with sporadic antibody availability are likely to experience a relapsing/remitting form of MS and milder disease. The relapse frequency is tied to the frequency of BBB disruption and/or to systemic antibody fluctuation. While the BBB remains intact, specific IgE is not available for mast cell degranulation nor are non-IgE antibodies available for complement fixation. New inflammation will not take place, and any existing inflammation will wind down. Patients with fairly constant antibody availability are expected to suffer from the chronic progressive form of MS. Their diseases are expected to progress at a faster rate and/or their neurological deficits are often more severe than experienced by RR patients.

Immunoassay for Detecting Specific IgE

A method for detecting a disease caused by or affected by IgE antibodies that complex with self-molecules in a subject. The IgE antibody in a biological fluid that is specific to a self-molecule is detected by an immunoassay. Such diagnostic test is useful in identifying early and subclinical disease, confirming a disease or monitoring a therapeutic treatment. The test sensitivity and specificity can be optimized by using a wider selection of test antigens, including intact proteins, glycoproteins, or pre-selected three-dimensional fragments thereof.

The biological fluid can be any fluid from a subject such as serum, plasma, whole blood, urine, sweat, saliva, etc. Preferred biological fluid is serum or plasma.

In one embodiment, the immunoassay for detecting the specific IgE comprises the steps of: (a) reacting the biological fluid with a solid phase immobilized with the antigen molecule to form an immunocomplex between the antigen and the specific IgE; (b) removing unbound materials from the solid phase; (c) reacting the immunocomplex-bound solid phase with an anti-IgE antibody labeled with a reporter molecule; (d) removing unbound materials; and (e) detecting a signal generated from the reporter molecule on the solid phase of (d).

Various solid support materials can be used for immobilization of an antigen by methods known to a skilled person. The solid support is insoluble in water; it can be rigid or non-rigid. The solid support includes filter paper, filtering devices (e.g., glass membranes), plastic beads (such as polystyrene beads), zest tubes or (multiple) test wells made from polyethylene, polystyrene, polypropylene, nylon, nitrocellulose, and glass microfibres. Also useful are paper discs (Schleicher and Schuell), and particulate materials such as agarose, cross-linked dextran and other polysaccharides. A peptide or protein antigen can be covalently or non-covalently bound to a solid support. For example, a peptide or protein antigen can be covalently bound to an activated solid support having reactive groups capable of forming covalent bonds with peptide or protein, such as CNBr-activated solid support, by conventional means. A peptide or protein antigen can be bound to polystyrene beads by non-specific adsorption. A peptide or protein antigen can also be bound covalently to polystyrene beads containing carboxyl or amino functional groups by conventional means.

The anti-IgE antibody that reacts with the immunocomplex of antigen and specific IgE is preferably a polyclonal anti-human IgE antibody. The antibody is labeled with a reporter molecule such as a radioactive isotope, an enzyme, biotin, avidin, a chromogenic substance, a fluorogenic substance, or a chemilumenescent molecule. The signal generated by the reported molecule immobilized on the solid support is proportional to the amount of the immunocomplex formed; therefore, the signal reflects the specific IgE level in the biological fluid.

Any immunoassay (e.g., radioimmunoassy, fluorescent immunoassy, enzyme immunoassay, and chemiluminescent immunoassasy) that provides proper sensitivity and specificity can be used to determine the specific IgE quantity. A preferred immunoassay is a radioimmunoassy modified from the procedures described in Ceska M. (Radioimmunoassay of IgE using paper disks. Methods Enzymol, 73(Pt B): 646-56 (1981)).

In one aspect, the invention includes a diagnostic kit for use in the methods described above. The kit includes an antigen-immobilized solid support, and detection means for detecting the amount of immunocomplex formed by the antigen and the specific IgE from a biological fluid sample.

Using an ultrasensitive blinded assay of high specificity, data show that anti-myelin protein-specific IgE is present in many patients with relapsing (relapsing-remitting [RR] and secondary-progressive [SP]) MS. These findings suggest that the allergic arm of the immune system plays a pivotal role in two distinct ways. First, blood-brain barrier (BBB) disruption may result from excessive, intravascularly disseminated histamine, a by-product of the conventional allergic diathesis. Second, toxic anti-myelin autoantibodies of the IgE isotype class may be produced. An antibody response subsequent to demyelination that is mediated by some other mechanism, or could be primary, which would indicate that, at least in a subset of MS patients, an early immune derangement could be triggered by the allergic arm of the immune system. In either case, autoreactive IgE may be pathogenic; persistence could be facilitated by the fact that IgE production generally lasts longer than other antibody isotypes, especially if the driving antigen is on myelin and adequate, supporting immune cells are locally available.

The size of a peptide required for individual antibody binding (the epitope) is approximately 5-6 amino acids in length. A synthetic peptide of this size can adequately substitute for a three dimensional epitope of a parent molecule because, given its small size and freedom to bend, it is able to precisely fit into the antigen binding cup of a specific antibody.

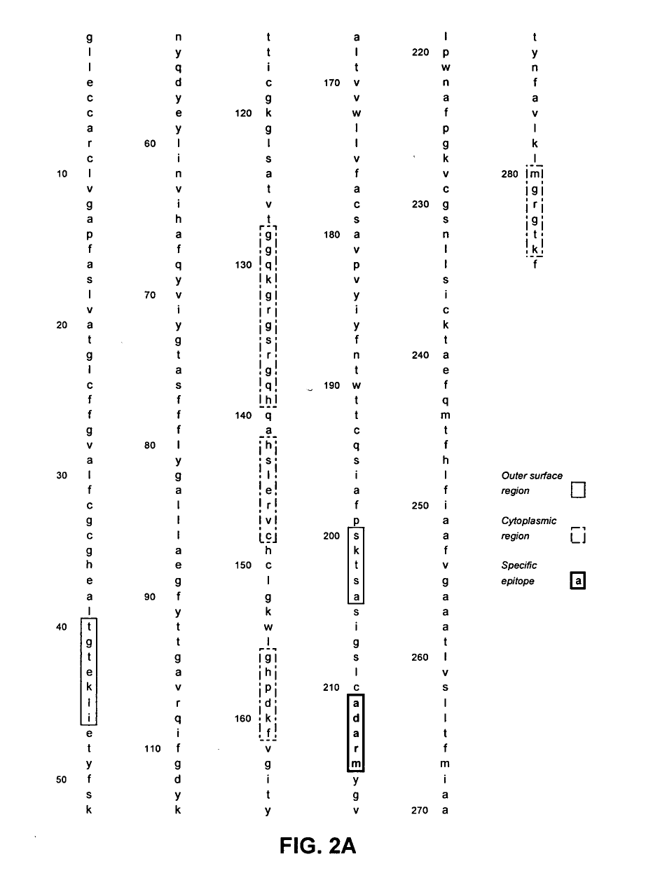

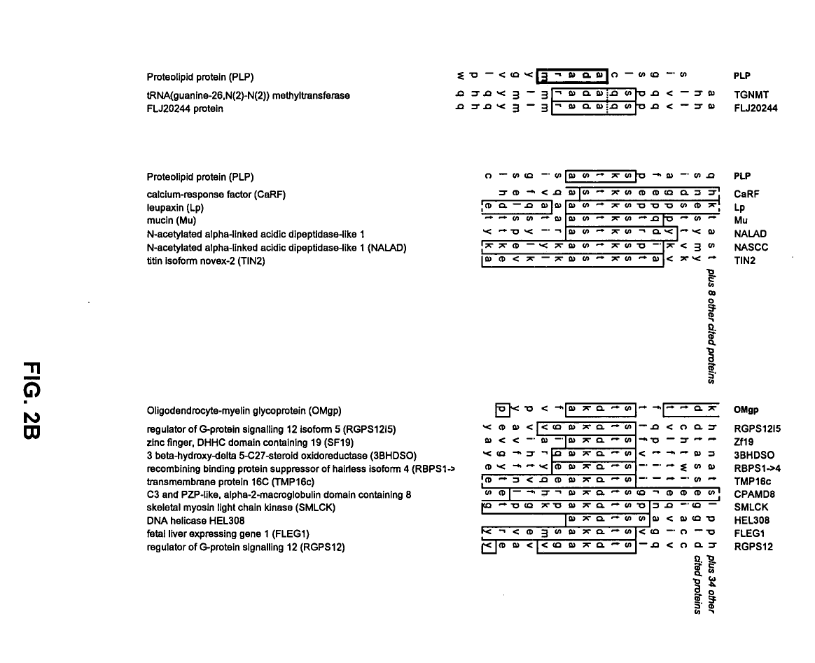

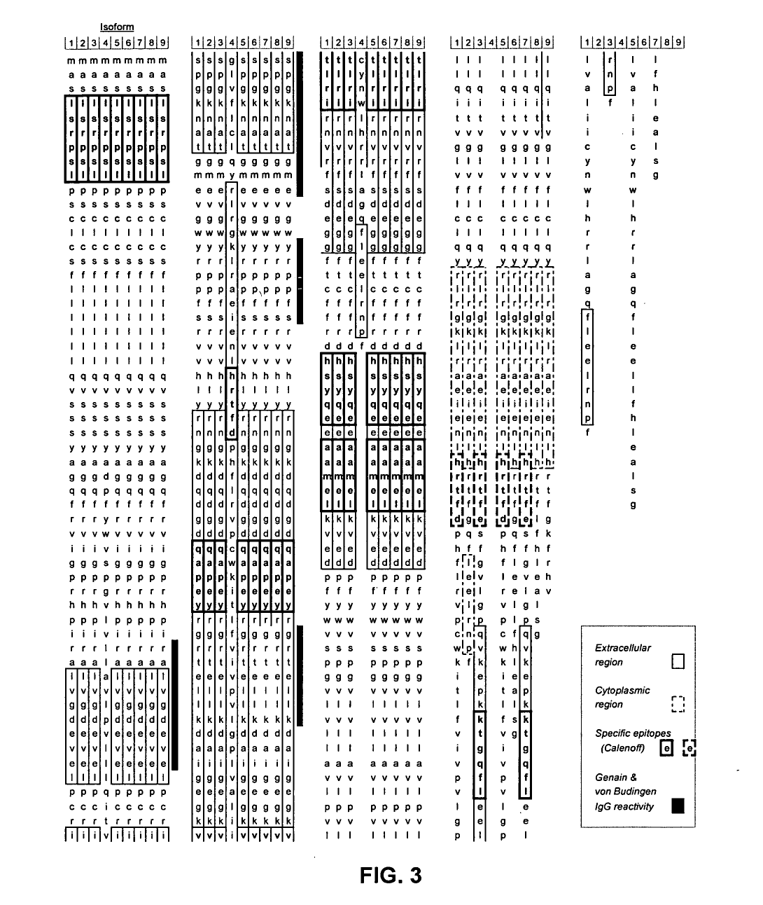

It is intuitive that the peptide structure of an epitope recognized by harmful autoantibodies must be sufficiently unique so as to escape homeostatic immune surveillance against faulty, self-directed immune reactivity. That is, a similarly structured peptide region found on diverse endogenous proteins would present multiple, simultaneous targets for a devastating, multi-organ immune attack. Organ-specific autoantibody production is more likely to be directed against peptide regions that share minimal structural overlap with peptide regions of other endogenous proteins. This point is illustrated by data regarding the pentameric sequence ADARM of PLP, which appears to be both an auto-Ig target and structurally unique among human proteins (FIGS. 2, 3). The complete pentameric sequence of this peptide is fully surface-exposed on PLP for specific and avid antibody coupling. It is less so (four contiguous, surface amino acids) for the sole other protein that shares the same 5-amino acid sequence (but with fewer sequential amino acids available for protein surface coupling). The ADARM sequence of PLP is therefore structurally and functionally unique, sharing little conservation with other proteins.

In order to serve as a functional epitopic antigen, a pentapeptide must usually represent a sequence that is fully exposed on the protein surface and is thereby accessible for specific 3D antibody coupling. Having a single amino acid out of sequential alignment likely throws off specific antibody binding as reflected in FIGS. 2 and 3 by the ADARM, SKTSA, and STDKA peptides. Under these guidelines, ADARM is functionally specific for PLP, whereas the SKTSA peptide regions are exposed on more than 1 human protein and are probably more restrictive from the point of view were they autoepitopes, they would be targeted at many different body sites with a more dire life and death consequence. From the evolutionary point of view, these sites would be less likely to become autoepitopes than sites that are more structurally unique and resulted in more focused, less immediately serious pathology.

The other tested pentameric sequences analyzed tested IgE-negative when used as source antigen and are also common, shared sequences among other proteins: SKTSA, derived from PLP and STDKA, derived from OMgp (FIG. 2B). Similar IgE-negative results have also been obtained with other common pentameric peptide sequences from MAG and MBP. When both structural and functional homology exist for a given peptide region that is shared among large numbers of different human proteins, a directed autoimmune response is unlikely. Conversely, a directed antibody response is more likely for unique peptide sequences contained within proteins that have restricted tissue expression.

Both harmful and beneficial autoantibodies may coexist, differing as to their antibody isotype and molecular targets. A beneficial autoantibody could include antibodies that aid in tissue repair, clearing of cellular debris, etc. It is therefore important to differentiate between distinct antibody isotypes, their target epitopes, and their potential pathogenicity in mapping out autoantibody processes associated with MS. Autoreactive IgE may be pathogenic for various reasons. Elucidation of the prevalence of anti-myelin IgE, its concentration and the kinetics of its appearance/disappearance over time, and the identification of disease-specific epitopes may shed light on the pathogenic potential of these antibodies and lead to new therapeutic strategies.

Blinded Allergy Screen of MS Patients and Controls

If an IgE response is relevant in MS, one would expect an increased occurrence of allergies in the MS population. MS patient (n=53) and healthy donor (n=30) sera were assayed for measurable anti-IgE to common allergens. The demographics for the 83 subjects tested is shown in Table I. The make-up of the MS sample approximates that of larger MS populations in cross-section.

TABLE I
Demograhics of MS and control subjects.

| | | | Mean | Mean | % |
|---|---|---|---|---|---|
| | n = | Female | Age | EDSS | Caucasian |
| Controls | 30 | 50.0 | 41.6 | NA | 93.3 |
| CIS | 3 | 100.0 | 25.0 | 1.5 | 100.0 |
| RRMS | 29 | 69.0 | 37.2 | 2.1 | 96.6 |
| SPMS | 14 | 71.4 | 49.8 | 5.9 | 92.9 |
| PPMS | 7 | 42.9 | 49.7 | 6.3 | 100.0 |

Table II. IgE Anti-Myelin Peptide Positive Subjects.

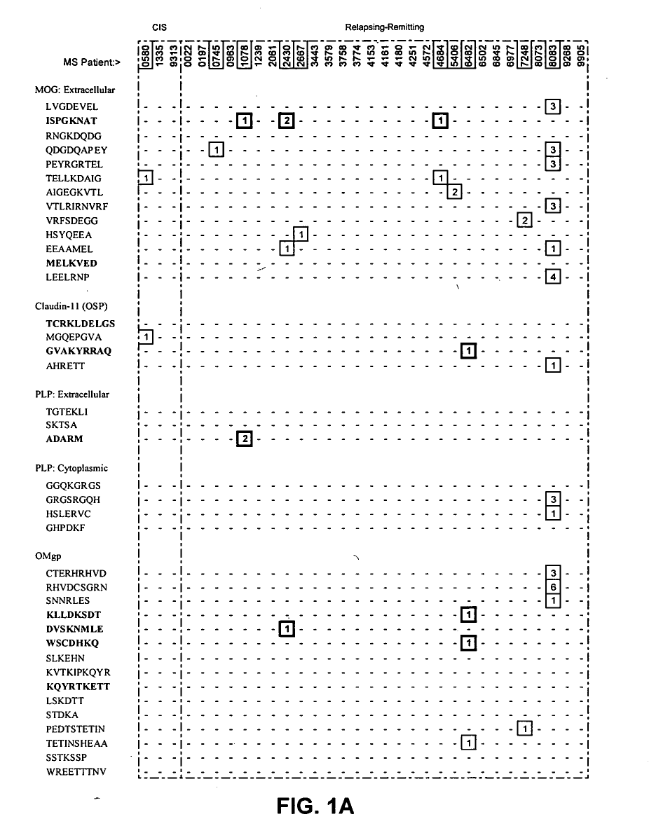

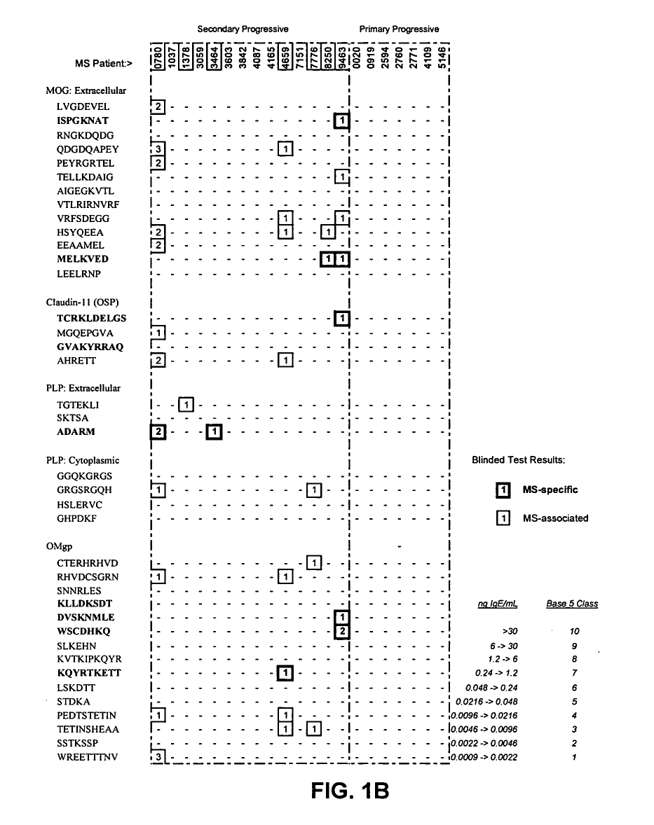

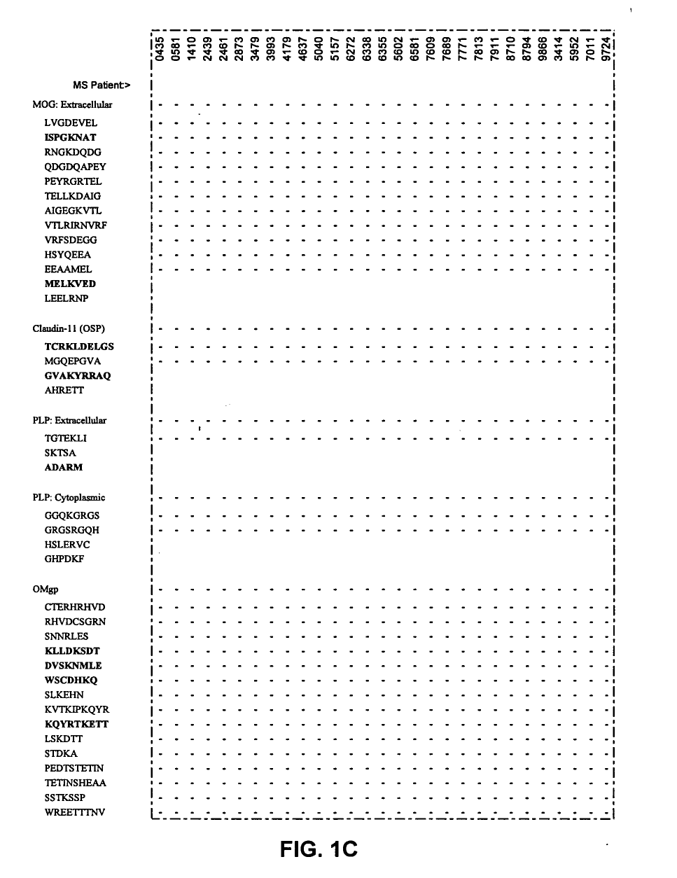

Total numbers and percentages of MS patients (by subtype) and controls testing positive for IgE against at least one of the screened myelin protein peptides are shown (see FIG. 1 for details). 50% of SPMS patients were positive, as compared to 31% of RRMS patients and 0% of PPMS patients.

*The sole positive control (5630) is a biological sister of a RRMS patient (1078) (see FIG. 1C).

IgE Specific for Distinct Myelin Protein (Peptide) Epitopes Detected in MS Patient Serum

Serum was denoted as IgE positive against a given myelin protein peptide if quantification of reactive IgE was >2.0× the standard deviation (SD) of the test value of a non-specific control peptide (FTISRDNAE, epsilon chain of human IgE) covalently coupled to paper discs employed in the modified eRAST assay. A second adjustment was subtraction of the mean of the control test values +2×SD corresponding to individual test peptides from those of MS patients in order to ascertain the net positive value (if any) for MS patients. At first glance, 17/53 (32%) MS patients' sera were IgE-positive against one or more myelin protein peptides, as compared to 1/30 control subjects, when tested blindly (Table II). It is of note that the sole positive control (5630) is a younger biological sister of one of the MS patients (1078), the latter of whom was one of the 17 positive patients. Excluding the 7 PPMS patients who are all negative for anti-myelin IgE, 17/46 (37%) relapsing (RRMS+SPMS) patients are positive. 10/32 (31%) RRMS patients (including 3 CIS) are positive, compared to 7/14 (50%) SPMS patients. Relapsing MS patients more often tested allergen positive as compared to PPMS patients or controls (bottom panels of FIG. 1A-C).

As shown in FIGS. 2A and B, 65 positive epitopes are identified on the 17 positive MS patients, all subtypes included, with MOG accounting for 30, OMgp 19, PLP 8, and OSP 8. When random serum samples were rescreened, identical confirmatory results were obtained. Of the 10 positive RRMS patients, 30 positive epitopes are identified (range 1-12 epitope hits per patient) while 35 positive epitopes are identified for the 7 SPMS patients (range 1-12 epitope hits per patient). Taking a closer look at these results, on average there are 2.5 epitope hits per SPMS patient and 0.94 in the RRMS group (including the 3 CIS patients). Of the positives in each group, only 3/10 positive RRMS patients exhibit >2 epitope hits, as compared to 4/7 in the SPMS group. Together, these data suggest that anti-myelin IgE correlates with progressive disease. The sole positive control (5630) had positive epitopes within MOG (3), OMgp (3) and PLP (3), but not OSP (FIG. 1C); her sister with RRMS (1078) had 2 positive epitopes. While patient #5630 could represent a false positive, it is also conceivable that this individual has preclinical MS (risk of developing MS in first-degree relatives is 2-4%).

As shown in FIG. 1B serum IgE positivity against myelin protein peptides is shown for 14 SPMS (left) and 7 PPMS patients tested (right).

As above in (a), a box indicates IgE reactivity against that peptide, with titer of specific IgE indicated by the number within the box (see key, below). Reactivity requires signal >2.0×SD above control peptide. In cases where peptide reactivity is unique to the tested MS population, boxes are stippled (suggesting disease specific peptide sequence); in cases where peptide reactivity is also found in controls but at much lower levels (>2.0×SD), boxes are highlighted without stippling (suggesting disease associated sequence).

Serum from SPMS patients more often tested IgE positive against one or more allergens, as compared to PPMS patients or controls (bottom).

FIG. 1C shows serum IgE positivity against myelin protein peptides for 30 controls. A box indicates IgE reactivity against that peptide, with titer of specific IgE indicated by the number within the box (see key, above). Reactivity requires signal >2.0×SD above control peptide.

IgE anti-myelin peptides could be pathogenic or an epiphenomenon, in which case they might nonetheless be an important biomarker. To be pathogenic, two IgEs would have to be closely spaced so as to cause mast cell degranulation. This situation could arise from either a single type of IgE binding to the same epitope on closely spaced molecules, or two closely spaced epitopes, defined as spacing between 40 and 120 angstroms on an individual protein surface. The occurrence of at least two adjacent IgE-immunoreactive peptides on the same myelin protein is seen several of the positive subjects, as shown in FIG. 1. It is of interest that the epitopes identified were commonly shared in patients, suggesting a common target epitope. The highest occurrence of IgE reactivity against the tested peptides was four (4 each for the MOG peptides ISPGKNAT, QDGDQAPEY and HSYQEEA).

While the sensitivity in this study appears low (31% for RRMS and 50% for SPMS) it is of high specificity vs. healthy controls, in contrast to other published serum assays. A number of potential explanations might explain the “low” sensitivity: 1) MS is heterogeneous, and not all MS patients may exhibit-the same aberrant immune mechanisms (specifically, anti myelin peptide Ig); 2) Ig of subtypes other than IgE might be relevant in “negative” cases; 3) Ig against additional untested target antigens might exist; or 4) Ig against native myelin protein epitopes (not represented as a linear peptide), carbohydrate, or lipid determinants might be relevant in “negative” cases. The fact that individual patients often have multiple IgE anti-myelin peptide “hot spots” suggests that no single epitope is likely to be unique to the disease process.

In summary, the importance of autoantibody in MS has been neglected until recently, and the significance and consequence of anti-myelin IgE is untested. Specific autoantibodies might contribute to CNS injury (pathogenicity) and/or might be a marker of disease. Methods and compositions described herein are diagnostic, and potentially therapeutic, relevance in MS. Assays are of high specificity and are abnormal in a subpopulation of multiple sclerosis patients. There is a possible biological differentiation between RRMS/SPMS and PPMS.

As shown in FIG. 2B, the pentameric sequence ADARM of PLP, appears to be both an auto-Ig target and structurally unique, sharing little conservation with other human proteins. Other tested pentameric sequences analyzed tested IgE-negative when used as source antigen and are also common, shared sequences among other proteins: SKTSA, derived from PLP and STDKA, derived from OMgp (FIG. 2B).

IgE Antibody-capture Immunoassay: (1) anti-human IgE antibody is covalently coupled onto the paper discs used in the eRAST format; (2) test sera is incubated onto the paper disc in order that all sample IgE is captured; (3) follow-on incubation of 1¹²⁵-labeled specific peptide is secondarily captured by epitope-specific, captured IgE; (4) radiolabel tracer antibody is detected in a Packard™ gamma counter.

IgA, IgG, IgG₁, IgG3, and IgM Antibody Assays: (1) neutravidin™ (NA) is covalently coupled onto the paper discs used in the eRAST format; (2) test sera plus biotinylated test peptide is separately incubated; (3) follow-on incubation of the serum solution of step 2 takes place on an NA disc; (4) incubation of 1¹²-labeled anti-IgA, IgG, IgGI, IgG3, and IgM; is incubated on the washed NA disc; (5) radiolabel is detected in a Packard™ gamma counter.

Ascertain from blinded analysis whether (a) specific antibody presence is associated with MS and/or (b) is indicative of MS pathology, e.g. IgE (capable of mast cell degranulation) and/or IgGI or IgG3 (capable of complement fixation).

IgE reactive to linear myelin peptide is a common finding in relapsing MS and is of high specificity vs. healthy controls. The number of IgE reactive epitopes may be associated with disease duration, which is agreement with an earlier study investigating serum IgG against MOG and MBP. Progression of disease has also been reported to correlate with antibodies against myelin proteins, CSF neurofilament light chain, and antibodies against neurofilament light chain.

Another tissue destructive situation involves complement fixation (with either IgGI, IgG3, and IgM). IgG identified as “positive” are relevant, as they are able to fix complement and can thereby result in CNS injury.

### EXAMPLES

### Example 1

### General Procedures for Preparing Covalent-Ready, Cyanogen Bromide Activated Paper Discs

Examples 1-3 are procedures modified from those described by Ceska (1981).

Materials

paper discs: Schleicher and Schuell 53870.

CNBr solution: 20 gm cyanogens bromide (CNBr, Sigma c6388) +600 mL distilled water.

1M NaOH.

0.05M NaHCO3.

25%, 50%, 75%, and 100% acetone.

Distilled Water.

Dessicant packets: Sigma S8394.

Zip lock plastic bags.

Procedures

The following procedures are performed under a hooded, well-ventilated environment. 20 gm paper discs are swelled in 200 mL distilled water at room temperature. Swelled paper discs are then added to 600 mL of CNBr solution while stirring. Bring up the pH of the stirring mixture to 10.5 and maintain at pH 10.5 until 100 mL of 1N NaOH have been used up. Aspirate the resulting liquid and wash discs with 500 mL of NaHCO3 buffer for 2 minutes at room temperature. Repeat wash step ×12. Rinse discs twice with 500 mL distilled water. Rinse discs twice with 500 mL 25% acetone. Rinse discs twice with 500 mL 50% acetone. Rinse discs twice with 500 mL 75% acetone. Rinse discs twice with 500 mL 100% acetone. Aspirate last acetone wash solution and allow discs to dry under a running fume hood at room temperature. Store dried CNBr activated paper discs in zip lock plastic bags containing dessicant packettes.

### Example 2

### Preparation of Peptide, Protein, or Protein Fragment Conjugated Paper Discs (Antigen-Coupled Discs)

Materials

8-10 amino acid length peptides with an 8-amino-3,6-dicyclohex-ylidine)-3-methylbutyl group linker at peptide amino terminal end.

CNBr-activated paper discs: prepared by method of Example 1.

Bicarbonate buffered saline (BBS): 0.05M NaHCO3 +0.15M NaCl, pH 7.2.

0.05M ethanolamine solution.

0.2M sodium acetate buffer, pH 4.0.

Paper disc incubation buffer: 0.5% human serum albumin +0.05M sodium phosphate +0.15M

NaCl+0.05% NaN3 +0.5% Tween20.

Procedures

Peptide solution preparation: Peptides are dissolved in BBS at a 120 μg/mL concentration.

Protein and protein fragment solution preparation: Proteins or protein fragments are dissolved in BBS at a 200 μg/mL concentration.

Covalent Coupling of Peptides or Proteins or Protein Fragments.

20 CNBr-activated discs are added to each mL of peptide or protein solution. Each peptide or protein/disc mixture is agitated for 16 to 18 hours at room temperature. Each solution surrounding the respective paper discs is aspirated and each set of discs are washed ×3 with BBS. The washed discs are immersed in 0.05M ethanolamine solution and agitated for 3 hours in order to block any unreacted CNBr sites. Each set of paper discs is then washed ×3 with the sodium acetate buffer. During the third step, the paper discs are incubated in the sodium acetate buffer for 30 minutes under gentle agitation. Each set of paper discs is then washed ×4 in BBS and then stored in the paper disc incubation solution at 4 C.

### Example 3

### Specific IgE Assay Method

Equipment and Materials

Antigen coupled paper discs.

Gamma counter.

I¹²⁵ labeled polyclonal, goat anti-human IgE (Vector Labs, Burlingame, Calif.).

Polypropylene test tubes.

I¹²⁵ diluent buffer: 5% horse serum +0.05M sodium phosphate +0.15M NaCl +0.05% NaN3+

0.5% Tween20.

Saran kitchen wrap.

Wash buffer: 0.05M sodium phosphate +0.15M NaCl +0.5% Tween20.

Assay Procedures

1. Place antigen coupled paper disc in polypropylene test tube.

2. Add 100 μl of patient serum to tube.

3. Make sure that paper disc is fully immersed in serum.

4. Cover top of tube with Saran wrap.

5. Incubate serum+disc at 25 degrees C. for 18 hours.

6. Aspirate serum.

7. Add 0.5 mL of wash buffer, let stand for 1 minute, and then aspirate. Repeat 6 times.

8. Add 50 μl of I¹²⁵ labeled polyclonal, goat anti-human IgE dissolved in diluent buffer so as to yield 40,000 counts per minute when counted in gamma counter (anti-IgE solution).

9. Make sure that paper disc is fully immersed in the anti-IgE solution.

10. Cover top of tube with Saran wrap.

11. Incubate anti-IgE solution and disc at 25° C. for 18 hours.

12. Aspirate anti-IgE solution.

13. Add 0.5 mL of wash buffer, let stand for 1 minute, and then aspirate. Repeat 6 times.

14. Transfer disc into fresh polypropylene test tube.

15. Insert tube into gamma counter and count radioactive emission for 2 minutes.

### Example 4

### Preparing and Selecting Antigenic Peptides of Myelin/Oligodendrocyte Proteins

A total of 164 peptides of 8-10 amino acids in length corresponding to nine myelin/oligodendrocyte proteins (peptide number denoted as n) were synthesized by Mimotopes Ltd. (Melbourne, Australia). The nine myelin/oligodendrocyte proteins are:

1. ceramide UDP-galactosyltransferase (CGT, n=17);

2. 2′,3′-cyclic nucleotide 3′-phosphodiesterase (CNP, n=34);

3. the CNS isomer of myelin-associated glycoprotein (L-MAG, n=28);

4. myelin-associated oligodendrocyte basic protein (MOBP, n=20);

5. myelin basic protein (MBP, n=19);

6. myelin oligodendrocyte glycoprotein isomers (MOG, n=20);

7. oligodendrocyte transmembrane protein (Claudin-11 or OSP, n=4);

8. oligodendrocyte myelin glycoprotein (OMG, n=15);

9. proteolipid protein (PLP, n=7).

Each peptide was conjugated to CNBr activated paper discs according to Example 2. The peptide-coupled disk was incubated with serum from a multiple sclerosis patient, and the specific IgE activity was measured according to Example 3. The following peptides were shown to have antigenic activities.

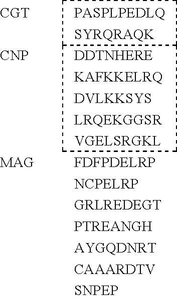

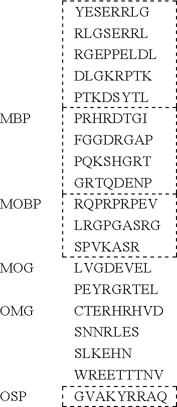

The peptides contained in the box are intracellular peptides; the other peptides are surface peptides. A surface peptide is likely to be more accessible to the specific IgE.

### Example 5

### Selecting IgE-Reactive Epitopes that Facilitates Mast Cell Degranulation

Not all IgE-reactive epitopes on an antigen molecule can facilitate mast cell degranulation. Those epitopes that facilitate mast cell degranulation are adequately situated so as to allow two or more specifically complexed IgE molecules to be: (1) spaced from between 40 to 100 angstroms apart; and (2) properly aligned such that their Fc domains for functional coupling with mast cell Fc receptors are adequately projected.

The following method identifies epitopes and epitope combinations that are able to facilitate mast cell degranulation.

Materials

paper discs possessing covalently-coupled, intact antigen molecules (Example 1).

paper discs possessing covalently-coupled antigen molecule fragments each containing specific

IgE reactive epitopes (Example 1).

paper discs possessing covalently-coupled peptides which are functionally representative of

individual IgE reactive epitopes (Example 1).

antigen-specific IgE test method described in Example 3.

MS patient serum samples

control samples from subjects without MS

Procedures

1. Patient and control serum samples are tested for specific IgE using paper discs containing and an intact antigen molecule.

2. IgE-positive patient and control sera are then retested using paper discs that possess corresponding, covalently-coupled antigen molecule fragments or peptides, each disc containing specific IgE reactive epitopes of the intact antigen molecule.

A. Second test results that do not indicate a mast cell degranulation:
a. Both patient and control sera are IgE positive against the same epitope. The epitope is expressed only once on the intact antigen molecule.
b. Both patient and control sera are IgE positive against the same epitope. The epitope is expressed more than once on the intact antigen molecule (polyvalent expression) in such a way so as not to be structurally available for functional IgE dimer formation.

c. Both patient and control sera are IgE positive against a single epitope but the reactive epitopes are different for each group. The corresponding intact molecule is not an isomer expressed in-vivo as a functional dimer or larger molecular complex.

d. The patients and control sera test IgE-positive for two or more epitopes on a single intact antigen molecule. The IgE-reactive epitopes are similar for both test populations and cannot effectively couple with specific IgE so as to form functional IgE dimers.

B. Second test results that indicate a mast cell degranulation:
a. Both patient and control sera are IgE positive against a single epitope on an intact antigen molecule but the reactive epitopes are different. The intact molecule is an isomer that is expressed in-vivo as a functional dimer or larger molecular complex. The patient group epitope is expressed on the surface of the intact antigen molecule in such a way so as to effectively complex with IgE and form functional IgE dimers or larger degranulating complexes. The control epitope is unable to couple with specific IgE so as to form degranulating IgE complexes.
b. Both patient and control sera are IgE positive against a single epitope on an intact antigen molecule but the reactive epitopes are different. The patient reactive epitope is expressed more than once on the intact antigen molecule (polyvalent expression). Each expressed epitope is available for complexing with two or more specific IgE molecules so as to form functional IgE dimers, trimers, or larger complexes. The control epitope is only expressed once.
c. The patients and control sera test IgE-positive against two epitopes on a single intact antigen molecule. One of the reactive epitopes is common to both groups. The second reactive epitope of each group is different. The patient reactive epitopes are able to effectively complex with available IgE so as to form a functional dimer. The control reactive epitopes cannot.
d. The patients and control sera test IgE-positive against two or more different epitopes on a single antigen molecule or on two or more functional adjoining antigen molecules. The patient reactive epitopes can couples with respective IgE molecules so as to form functional IgE dimers, trimers, or larger complexes. The control reactive epitopes cannot.

### Example 6

### Specific IgE and Allergy Test for MS Patients and Control Subjects

Twenty-four MS patients of different types and 12 control subjects were tested for specific IgE against antigenic peptides of myelin/oligodendrocyte (see Example 4). The 12 control subjects were from eight allergic patients with chronic asthma, varying in severity from mild to severe, and four patients with chronic allergic rhinitis. All control patients were negative for a history of neurological illness except for patient 4, an asthmatic who had a prior history of epileptic seizures. None of the control subjects were being treated with systemic steroids or with other medication known to alter IgE antibody production.

The specific IgE assay was performed according to Example 3. All positive data points and a representative number of test-negative data points were retested. The repeated analysis confirmed the validity of the initial test results.

In order to identify the coexisting allergy, MS patients were also screened for allergen-specific IgE against three different pollens (Ky. blue grass, oak tree, and short ragweed), D. farinae mite, Alternaria tenuis mold, and bovine milk. The allergy test was performed according to method of Ceska (2001). A log 5 scoring system was employed wherein: Class 1 described IgE levels in the 4.8 to 24 pg/mL range; Class 2 was 25 to 120 pg/mL; Class 3 was 121 to 600 pg/mL; Class 4 was 601 to 3,000 pg/mL; and Class 5 was 3,001 to 15,000 pg/mL. The IgE and allergy results for the MS patients and control subjects are summarized in Tables 2a and 2b, respectively.

Seventeen MS patient sera were IgE-positive against one or more myelin protein peptides (Table 2a) as compared with six control subjects (Table 2b). Among MS patients, positive reactions were elicited against peptides from CGT, CNP, MAG, MBP, MOBP, MOG, OMG, OSP and MOG, and recombinant ECD. In comparison, control subjects were only marginally IgE-positive against MAG, MBP, MOBP, and recombinant ECD, and no control sera were positive against CNP peptides.

The cerebrospinal fluids (CSF) of the MS patients were tested for specific IgE. There was not specific IgE detected

No apparent difference in IgE reactivity was noted among patients with relapsing/remitting MS as compared with patients with chronic progressive disease. Also absent were differences based upon the type of MS treatment or upon the absence of treatment.

All MS serum samples were tested positive for allergy. All patients were IgE-positive against bovine milk, and nineteen patients were also positive against one or more inhalant allergens.

Five MS patients possessed IgE/specific epitope combinations likely to induce mast cell degranulation on the outer surface of CNS myelin and oligodendrocytes. Four possessed IgE against a single epitope of MOG, a protein known to exist as a dimer or trimer (Salvin, 1997) and one patient was reactive to two individual OMG peptide sites.

### Example 7

### Specific IgE Assay Against Intact MOG Complexes

In order to evaluate whether an intact protein, with its three dimensional structure and greater number of potential epitopes, is more useful than a peptide as a test antigen, the 24 MS sera and 12 control sera (see Example 6) were tested for specific IgE reactivity toward murine non-glycosylated, recombinant extracellular domain MOG (Kindly provided by Dr. Minnetta V. Gardinier, Dept. Pharmacology, School of Medicine, University of Iowa, Iowa City, USA), which has about 95% homology to human MOG protein. Eight of the MS patient sera and one control serum tested IgE positive (see Table 2a and 2b: MOG Recombinant ECD). The MS patients exhibited much higher MOG-specific IgE levels than did the control. The lone control subject (patient 4) with a positive MOG-specific IgE level had a prior history of epileptic seizures.

Overall, the specific IgE levels detected when using intact murine MOG as a test antigen were significantly higher than by using small MOG-derived peptide antigens that corresponded to human MOG. Several serum samples that were IgE negative against the murine MOG were IgE positive when tested against peptides corresponding only to human MOG, said peptide regions being absent or significantly altered in the mouse version of MOG. Aside from the structural differences between human and murine MOG, and for attaining greater sensitivity and sensitivity, intact protein appears to be a better antigen than short, corresponding peptide.

### Example 8

### Analysis of the IgE-Reactive Peptides

The IgE-reactive peptides of mylin/oligodendrocytes were analyzed using the BLAST search tool of the National Library of Medicine. The results show that most of the reactive peptides were significantly similar in structure to peptide regions of bacterial, food (Stefferl, 2000), fungal, or viral proteins (Table 3), which are foreign proteins to human. These results may explain how specific IgE's against mylin/oligodendrocytes peptides are developed in patients even though mylin/oligodendrocytes proteins are behind the blood-brain barrier.

TABLE 3

Similarity of Myelin/Oligodendrocyte
Peptide to Extraneous Protein Source

| | |
|---|---|
| Protein Source | Peptide |

| | |
|---|---|
| CNP | DDTNHERE |
| Tobacco | DDSNHER |
| CNP | KAFKKELRQ |
| Neurospora crassa | KAFKKEL |
| CNP | DVLKKSYS |
| Rice | DVLKKAY |
| CNP | LRQEKGGSR |
| Fusobacterium nucleatum | LRQEKG |
| CNP | VGELSRGKL |
| Pseudomonas aeruginosa | VGEFSRGK |
| MBP | PRHRDTGI |
| Pseudomonas aeruginosa | _HRDTGI |
| MBP | FGGDRGAP |
| Rice | FGGDRG |
| MBP | PQKSHGRT |
| Clostridium perfringens | QKSHGR |
| MBP | GRTQDENP |
| Bacillus subtilis | RSQDENP |
| MOBP | RQPRPRPEV |
| Salmonella enteritidis | _PRPRPE |
| MOBP | LRGPGASRG |
| Pseudomonas aeruginosa | LRGPGATR |
| MOBP | SPVKASR |
| loblolly pine lacase | SPVKATR |
| MOG | LVGDEVEL |
| Leptospira interrogans | _DEVEL |
| MOG | PEYRGRTEL |
| Pseudomonas aeruginosa | _RGRTEL |
| OMG | CTERHRHVD |
| Pseudomonas putida | _TERHRH |
| OMG | SNNRLES |
| Saccharomyces cerevisiae | SNNRLE |
| OMG | SLKEHN |
| Mycoplasma pneumoniae | SLKEHN |
| OMG | WREETTTNV |
| Staphylococcus aureus | WREETT |

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude this specification.

Materials and Methods

Myelin Protein Peptides and Anti-Myelin Peptide Radioimmunoassay

The basic method of Ceska was modified to use sequential or overlapping peptides made up of 5-10 amino acids (for the most part 8-10-mers), derived from the known primary structures of human myelin oligodendrocyte glycoprotein (MOG), oligodendrocyte-myelin glycoprotein (OMgp), proteolipid protein (PLP), or claudin-11 (oligodendrocytes-specific protein, OSP), as source antigen to quantify peptide-specific IgE in the range of 0.05-60 pg/ml. Peptides were synthesized by Mimotopes Ltd., Melbourne, Australia with an 8-amino-3,6-dicyclohexylidine-3-methylbutyl group linker.

Peptides were dissolved in bicarbonate buffered saline (BBS), pH 7.2 at a 120 μg/mL concentration. CNBr activated paper discs were mixed together with 1 mL of dissolved peptide solution and processed by the method of Ceska. IgE reactive to individual epitopes (tested peptide number denoted as n) of extracellular MOG (n=13), OSP (n=4 ), OMgp (n=15), or PLP (n=7) was quantified (see Table III) in a highly sensitive radioimmunoassay.

Conventional Allergy Screening of Sera

In order to demonstrate serological evidence of allergy, subjects were screened for allergen-specific IgE against three different pollens (Ky. blue grass, oak tree, and short ragweed, D. farinae mite, Alternaria tenuis mold, and bovine milk) using eRAST (enhanced method of Ceska).

A standard log 5 scoring system was employed wherein Class 1 denotes IgE levels in the 4.8-24 pg/mL range, Class 2 25-120 pg/mL, Class 3 121-600 pg/mL, Class 4 601-3,000 pg/mL, and Class 5 3,001-15,000 pg/mL. Allergy test results are depicted in FIG. 1 as plus (Class I or above) or minus. All positive data points and a representative number of test-negative data points were retested. Repeat analysis confirmed the validity of the initial test results.

A humoral autoimmune peptide epitope, likely consists of 5-6 amino acids. As shown in FIG. 1B, among pentameric peptides thus far tested, only in the case of PLP's ADARM is the amino acid sequence both distinctive and exposed (for adequate antibody binding) whereas for SKTSA and STDKA, a large number of human proteins contain the identical sequence. This suggests that autoantibodies are likely to form when a given peptide is mimicked by a foreign immunogen and its structure is sufficiently unique so as to escape immune surveillance.

GenBank analysis of peptides (5-6 amino acids in length) derived from other tested myelin proteins reveals the presence of other potential disease-specific epitopes buried within OSP (n=1), MOG (n=6), and OMgp (n=7). These unique pentameric sequences are contained within somewhat larger, disease-specific or associated peptides already tested. From GenBank analysis of the primary structures of other candidate autoantigens, it appears that the following myelin proteins also possess pentameric or hexameric peptides that are similarly unique: MAG (n=7); unmodified MBP (n=4); and citrullinated MBP (n=6).

### PUBLICATIONS CITED

The publications listed below are incorporated by reference to the extent they relate to the materials and method disclosed herein.

Ceska M. Methods Enzymol, 73(Pt B):646-56 (1981).

Slavin, A. J., et al., Neurosci, 19:69-78 (1997)

Stefferl A., et al., J Immunol, 165:2859-65 (2000)

U.S. Pat. App. 2002/0193295

1. A method for detecting a disease in a subject caused or affected by antibodies that complex with self-molecules in the subject, the method comprising the steps of:
(a) identifying an antigen molecule on a self molecule of the subject comprising one or more epitopes, wherein the antigen molecule is specific to the disease and each epitope comprises five to six contiguous amino acids the sequence of which confers three dimensional target specificity for an individual antibody; and
(b) detecting a specific immunoglobulin antibody in a biological fluid of the subject, wherein the antibody forms an immunocomplex with said one or more epitopes on the antigen molecule whereby said disease is detected.

2. The method of claim 1, wherein the biological fluid is selected from the group consisting of plasma, serum, cerebrospinal fluid and whole blood.

3. The method of claim 1, wherein the specific immunoglobulin is selected from the group consisting of IgA, IgD, IgE, IgG, and IgM.

4. The method of claim 3, wherein the specific IgA antibody is IgA1.

5. The method of claim 4, wherein the specific IgA2 antibody is pathologic by being able to fix complement.

6. The method of claim 4, wherein the specific IgA1 antibody is protective by being able compete with co-existing, harmful antibodies or T lymphocytes for specific epitope binding or by supplanting specific epitope binding by harmful antibodies or lymphocytes.

7. The method of claim 3, wherein the specific antibody is IgG.

8. The method of claim 7, wherein the specific IgG antibody is selected from the group consisting IgG1 and/or IgG3 and is pathologic by being able to fix complement.

9. The method of claim 7, wherein the specific antibody is IgG2 and/or IgG4 and is protective by being able compete with co-existing, harmful antibodies or T lymphocytes for specific epitope binding or by supplanting specific epitope binding by harmful antibodies or lymphocytes.

10. The method of claim 1, wherein the specific antibody is IgM able to serve as an opsonizing agent for cellular autoimmunity.

11. The method of claim 1, wherein said antigen molecule is selected from a group consisting of a peptide, a protein, and three-dimensional protein fragments.

12. The method of claim 1, where said disease is an autoimmune disease, a neurological disease, or a psychiatric disease.

13. The method of claim 12, wherein the autoimmune disease is multiple sclerosis and the self-molecule is a constituent of an oligodendrocyte or central nervous system myelin.

14. The method of claim 13, wherein the self-molecule is selected from the group consisting citrullinated myelin basic protein, Claudin-11, myelin associated glycoprotein (MAG), myelin basic protein (MBP) and myelin oligodendrocyte basic protein (MOBP).

15. The method of claim 3, wherein:
(a) IgE forms an immunocomplex with one or more epitopes in the form of an IgE dimer, IgE trimer, or IgE oligomer;
(b) the immunocomplex is able to induce mast cell degranulation; and
(c) the mast cell degranulation initiates, sustains, or augments a specific disease process.

16. The method of claim 15, wherein the distance between each IgE molecule of said IgE dimer, IgE trimer, or IgE oligomer complex is between 40 to 100 angstroms.

17. The method of claim 15, wherein each IgE molecule of said IgE dimer, IgE trimer, or IgE oligomer complex is spatially arranged so as form an immunocomplex with Fc RI receptors on a mast cell to induce mast cell degranulation.

18. The method of claim 17, wherein the Fc portion of the IgE molecule has undergone a structural modification to facilitate its immunocomplex formation with Fc RI receptors on a mast cell.

19. A method for detecting a disease caused or affected by T lymphocytes that complex with self-molecules in a subject, the method comprising the steps of:
(a) identifying an antigen molecule comprising one or more epitopes on a self molecule of a subject, wherein the antigen molecule is specific to the disease and each epitope comprises five to six contiguous amino acids the sequence of which confers linear target specificity for an individual T lymphocyte;
(b) forming an immunocomplex between an epitope on the antigen molecule and the specificT lymphocyte;
(c) detecting a specific T lymphocytes in a biological fluid of the subject, wherein said lymphocytes are stimulated to proliferate in the presence of their corresponding specific epitope whereby said autoimmune disease is detected.

20. A plurality of immunogenic peptides of a self molecule, said peptides which produce a disease or condition specific immune response in a host, wherein the parent protein is causative of, or associated with, the disease or condition, and said peptides comprise the following structure:
(a) from 5 to 10 amino acids in length;
(b) an amino acid sequence which is identical to a contiguous amino acid peptide region of the sequence of a protein designated the parent protein;
(c) a net hydrophilic structure as determined by the amino acid sequence of the peptide, said structure located on the surface of the parent protein;
(d) an amino acid net sequence homology of 50 percent or less as compared with contiguous amino acid sequences of a comparative protein;
(e) an amino acid sequence wherein no more than three contiguous amino acids are identical to contiguous amino acids of the comparative protein matched for overall homology; and
(f) an antigenic profile which elicits an immune response specific for the parent protein as determined by results of immune cell proliferation assays or immunoassays of the disease or condition positive biological fluids compared to disease or condition negative biological fluids.

21. A pharmaceutical composition comprising the peptide ADARM (SEQ ID NO: 20).

22. A pharmaceutical composition comprising a plurality of immunogenic peptides of claim 20.

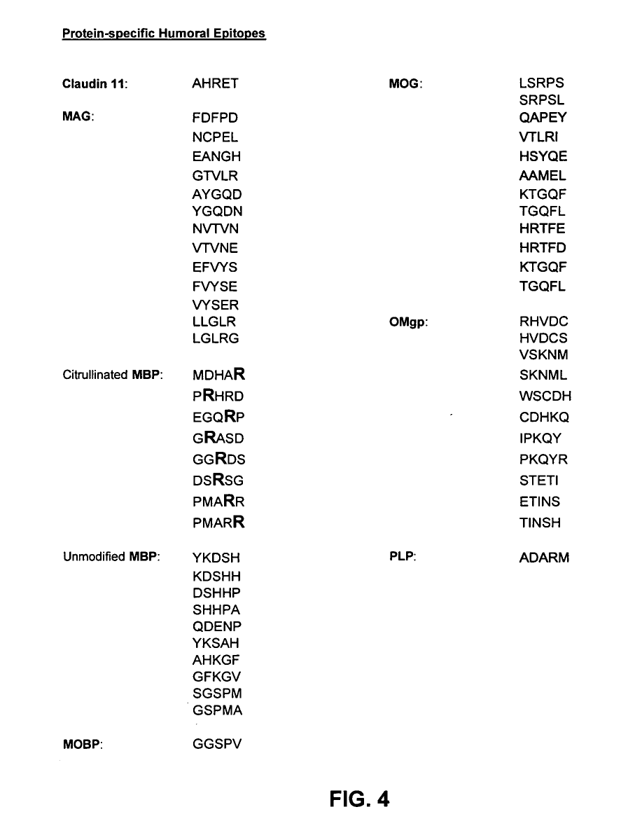

23. The pharmaceutical composition of claim 22 comprising peptides selected from the group of peptides in FIG. 4.

24. A method for identifying a peptide which functions as a highly specific antigen for a self molecule, said method comprising:
(a) selecting amino acid sequences of peptides of from 4 to 100 amino acids in length by copying the amino acid sequence of parent protein wherein the sequence satisfies the criteria of steps (a) to (c) of claim 20;
(b) synthesizing candidate peptides that have the sequences of step (a);
(c) labeling the peptides at either the NH2 or COOH end of their amino acid sequence with a detectable label; and
(d) testing by means of immunoassays whether the peptides are specific for the target protein.

25. A desensitizing reagent comprising the plurality of peptides of claim 20, said reagent used to down-regulate a specific immune response administered to a host affected with a targeted disease:
(a) in initial doses too weak to up-regulate causing immune response the disease; and
(b) incrementally increasing the dosage to induce immune tolerance to a specific antigen causing the disease thereby abrogating or ameliorating the disease process.

26. A method of inhibiting binding of antibodies to at least one antigen molecule selected from the group consisting of MOG, OSP, PLP, OMgp, MAG, MBP, and MOBP, the method comprising:
(a) obtaining a peptide made by the method of claim 24; and
(b) contacting a mixture of the antigen molecule with the peptide.

27. The method of claim 26, wherein the peptide is selected from the group consisting of peptides designated in FIG. 4.

28. The method of claim 27, wherein the peptide is ADARM (SEQ ID NO. 20).

29. A method for treating an autoimmune disease in a subject comprising the steps of:
(a) identifying one or more epitopes on a self molecule of a subject that are specific to an autoimmune disease;
(b) quantifying a specific immunoglobulin level in a biological fluid of the subject, wherein said immunoglobulin complexes with said one or more epitopes, an epitope comprising five to six contiguous amino acids the sequence of which confers three dimensional target specificity for an individual antibody;
(c) establishing a starting dose of a therapeutic antigen for desensitizing treatment, wherein said therapeutic antigen comprises said one or more epitopes; and said dose is inversely proportional to the quantitated immunoglobulin level of (b);
(d) administering to the subject the starting dose (c) of the therapeutic antigen; and
(e) administering to the subject an incremental dose of the therapeutic antigen over a period of time, to a level sufficient to down-regulate an immune process responsible for producing the immunoglobulin; whereby the symptom or physical finding of autoimmune disease is ameliorated.

30. The method according to claim 29, wherein the self-molecule is citrullinated myelin basic protein, Claudin-11, myelin associated glycoprotein, myelin basic protein, myelin oligodendrocyte basic protein, myelin oligodendrocyte glycoprotein, oligodendrocyte myelin glycoprotein.

31. A method for treating an autoimmune disease in a subject comprising the steps of:
(a) identifying one or more epitopes on a self molecule of a subject that are specific to an autoimmune disease, wherein an epitope comprises five to six contiguous amino acids the sequence of which confers linear target specificity for an individual T helper lymphocyte;
(b) identifying the presence of epitope-specific T helper lymphocytes in the subject's blood or other bodily reservoir by epitope-specific proliferation studies or by other studies;
(c) testing the expanded, epitope-specific T lymphocytes in cytoxicity studies directed against oligodendrocytes or other neural cells in order to confirm specific pathology;
(d) quantifying a specific immunoglobulin level in a biological fluid of the subject, wherein said immunoglobulin complexes with the epitope(s) specifically recognized by the T lymphocyte;
(e) establishing a starting dose of a therapeutic antigen for desensitizing treatment, wherein said therapeutic antigen comprises said one or more epitopes; and said dose is inversely proportional to the quantitated immunoglobulin level of (d);
(f) administering to the subject the starting dose (c) of the therapeutic antigen; and
(g) administering to the subject an incremental dose of the therapeutic antigen over a period of time, to a level sufficient to down-regulate an immune process responsible for producing the epitope-specific T lymphocyte; whereby the symptom or physical finding of autoimmune disease is ameliorated.
